(19) United States
(12) Patent Application Publication
(10) Pub. No.: US 2018/0342312 A1
(43) Pub. Date: Nov. 29, 2018

(54) METHOD AND SYSTEM FOR DIRECT ACCESS TO MEDICAL PATIENT RECORDS

(71) Applicant: Christopher Khatchig Kaypekian, Glendale, CA (US)

(72) Inventor: Christopher Khatchig Kaypekian, Glendale, CA (US)

(21) Appl. No.: 15/989,940

(22) Filed: May 25, 2018

Related U.S. Application Data
(60) Provisional application No. 62/511,748, filed on May 26, 2017.

(51) Int. Cl. G16H 10/60; G06Q 30/04; G06F 17/30; G06F 21/62

(52) U.S. Cl. CPC G16H 10/60; G06F 21/6218; G06F 17/30554; G06Q 30/04

(57) ABSTRACT

Systems for accessing patient medical records may comprise a frontend system, a log subsystem, a direct access system, the direct access system includes a content management subsystem for document intake creation and database storage, a report management subsystem for setting appointments and generating reports, a billing subsystem for generating billing and incidents reports, and a user access subsystem for user account management and access restrictions.

### CROSS-REFERENCE TO RELATED APPLICATIONS

This application claims priority to and the benefit of U.S. Provisional Patent Application No. 62/511,748, filed May 26, 2017, the contents of which are hereby incorporated by reference herein for all purposes.

### FIELD OF ENDEAVOR

The present embodiments relate to medical records. In particular, the present embodiments relate to accessing medical patient records in a direct access system.

### BACKGROUND

Medical information relating to a patient's care has been collected for centuries. This information that is contained in a medical record allows a patient's health care providers to quickly learn the patient's prior medical history, and thereby provides a high level of continuity of care to the patient. This medical record may also serve several other functions, such as providing a basis for planning the patient's future care, and documenting important communication between the patient's primary health care provider and any other health professionals that may be contributing to the patient's care. In some cases, the medical file can protect the legal interest of the patient and the health care providers responsible for the patient's care, and provides historical documentation of the care and services provided to the patient.

Traditionally, medical records have been written on paper and kept in folders. While these paper records have sufficed for some time, the creation and maintenance of paper files is extremely time consuming, particularly since these files are extremely detailed, and are often repetitive between patients resulting in duplicate efforts by the physician and his staff.

### SUMMARY

The various embodiments of the present medical records direct access systems have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

An embodiment may include a system for accessing patient medical records comprising, a frontend system, a log subsystem, a direct access system, the direct access system includes a content management subsystem for document intake creation and database storage, a report management subsystem for setting appointments and generating reports, a billing subsystem for generating billing and incidents reports, and a user access subsystem for user account management and access restrictions.

In a further embodiment, a content management subsystem also includes a patient information module, a document intake module, an attorney database module, an insurance company database module, and a physician database module.

In a still further embodiment, a report management subsystem also includes an incident information module, a report template module, an appointments module, and a patent report module.

In a yet still further embodiment, the billing subsystem also includes a billing codes module, an accounts receivables module, an incident invoice module, and a PDF generation module.

In an additional embodiment, the user access subsystem also includes a user account module, a user role module, and a user permission policy module.

In a still additional embodiment, the document intake module adds a new patient to the database, accesses the new patient, selects a document intake mode for the new patient, selects a document for the new patient to complete, generates a document from a template based on the selected document, displays the generated document, provides a method of user input on the document on the display including a method of indicating completion, generates a portable document format (PDF) document of the displayed document based upon a received response of completion, store the generated PDF document in a patient medical record associated with the new patient, mark the stored PDF document as complete in the in the patient medical record.

In a still additional embodiment, the patient reporting module authorizes a user onto the system, accesses a patient record from a database, determines type of report to be generated, generates questions for user based on determined type of report, receives report answer data from user, stores received report answer data to the database, generates a preliminary report, presents the generated preliminary report to the user, generates a finalized report, and transmits a message to a third party based upon the generated finalized report.

### BRIEF DESCRIPTION OF THE DRAWINGS

The various embodiments of the present medical records direct access systems now will be discussed in detail with an emphasis on highlighting the advantageous features. These embodiments depict the novel and non-obvious medical records direct access systems shown in the accompanying drawings, which are for illustrative purposes only. These drawings include the following figures, in which like numerals indicate like parts:

FIG. 1 is a conceptual block diagram illustration of a direct access system for patient medical records in accordance with embodiments of the invention;

FIG. 2 is a flowchart for accessing a patient in accordance with embodiments of the invention;

FIG. 3 is a flowchart for requesting an appointment in accordance with embodiments of the invention;

FIG. 4 is a flowchart for creating a report in accordance with embodiments of the invention;

FIG. 5 is a flowchart for processing intake documents in accordance with embodiments of the invention;

FIG. 6 is a flowchart of a general process overview of the medical records direct access system in accordance with embodiments of the invention;

FIG. 7A is a flowchart of an overview of an incident module in accordance with embodiments of the invention;

FIG. 7B is a flowchart of an overview of an billing module in accordance with embodiments of the invention;

FIG. 8 is a representative screen of a patient information module in accordance with an embodiment of the invention;

FIG. 9 is a representative screen of a patient information module in accordance with an embodiment of the invention;

FIG. 10 is a representative screen of a patient information module in accordance with an embodiment of the invention;

FIG. 11 is a representative screen of a patient information module in accordance with an embodiment of the invention;

FIG. 12 is a representative screen of a patient information module in accordance with an embodiment of the invention;

FIG. 13 is a representative screen of a document intake module in accordance with an embodiment of the invention;

FIG. 14 is a representative screen of a document intake module in accordance with an embodiment of the invention;

FIG. 15 is a representative screen of a document intake module in accordance with an embodiment of the invention;

FIG. 16 is a representative screen of a document intake module in accordance with an embodiment of the invention;

FIG. 17 is a representative screen of a document intake module in accordance with an embodiment of the invention;

FIG. 18 is a representative screen of an incident information module in accordance with an embodiment of the invention;

FIG. 19 is a representative screen of an incident information module in accordance with an embodiment of the invention;

FIG. 20 is a representative screen of an incident information module in accordance with an embodiment of the invention;

FIG. 21 is a representative screen of an incident information module in accordance with an embodiment of the invention;

FIG. 22 is a representative screen of a report template module in accordance with an embodiment of the invention;

FIG. 23 is a representative screen of a report template module in accordance with an embodiment of the invention;

FIG. 24 is a representative screen of a report template module in accordance with an embodiment of the invention

FIG. 25 is a representative screen of an appointments module in accordance with an embodiment of the invention;

FIG. 26 is a representative screen of an appointments module in accordance with an embodiment of the invention;

FIG. 27 is a representative screen of an appointments module in accordance with an embodiment of the invention;

FIG. 28 is a representative screen of an appointments module in accordance with an embodiment of the invention;

FIG. 29 is a representative screen of a billing codes module in accordance with an embodiment of the invention;

FIG. 30 is a representative screen of an incident invoice module in accordance with an embodiment of the invention;

FIG. 31 is a representative screen of an incident invoice module in accordance with an embodiment of the invention;

FIG. 32 is a representative screen of an incident invoice module in accordance with an embodiment of the invention;

FIG. 33 is a representative screen of an incident invoice module in accordance with an embodiment of the invention;

FIG. 34 is a representative screen of a user access subsystem in accordance with an embodiment of the invention,

FIG. 35 is a representative screen of a user access subsystem in accordance with an embodiment of the invention;

FIG. 36 is a representative screen of a log subsystem in accordance with an embodiment of the invention;

FIG. 37 is a representative screen of a front end subsystem in accordance with an embodiment of the invention; and

FIG. 38 is a conceptual illustration of a medical records direct access systems server in accordance with an embodiment of the invention.

### DETAILED DESCRIPTION

The following detailed description describes the present embodiments with reference to the drawings. In the drawings, reference numbers label elements of the present embodiments. These reference numbers are reproduced below in connection with the discussion of the corresponding drawing features.

Embodiments disclosed herein provided computer implemented method and system for direct access to a medical patient records, reports and billing by interested parties such as by attorneys, other practitioners, and insurance entities. These records are requested such as by fax, email or phone call which is usually handled by one or multiple staff members on both ends.

One embodiment provides a direct access system that enables a patient, a doctor's office or an attorney's office to make an appointment via a web portal of the direct access system. A unique identifier such as an identification number is assigned to each patient. A case number may also be assigned at that time.

Information about medical appointments and/or the patient can be transmitted securely. Once a patient arrives and checks in with the front office staff, a message will be automatically transmitted by the direct access system via email and/or text to the referring practitioner, attorney, and/or insurance representative (adjustor, etc.) stating that the patient has arrived and was evaluated and/or had undergone a procedure. A missed appointment notification will also be transmitted by the direct access system if the patient does not show up for their scheduled consultation, follow up visit, and/or procedure.

Following the patient's appointment, a password-protected and/or encrypted report is generated and uploaded into the direct access system, together with the charge for the visit and/or the procedure. This password-protected and/or encrypted report may be accessed at any time by using an access device such as computer, mobile computing device, tablet, smartphone, etc. by anyone who is allowed to have access.

Up-to-date and essentially instantaneous information is available regarding a patient's care which may be accessed by a user remotely by access to the direct access system (e.g., via internet) as long as the user is given specific access to password-protected and/or encrypted patient information which is compliant to HIPPA rules and regulations. The direct access system provides access to patient information including subpoena of records and billing.

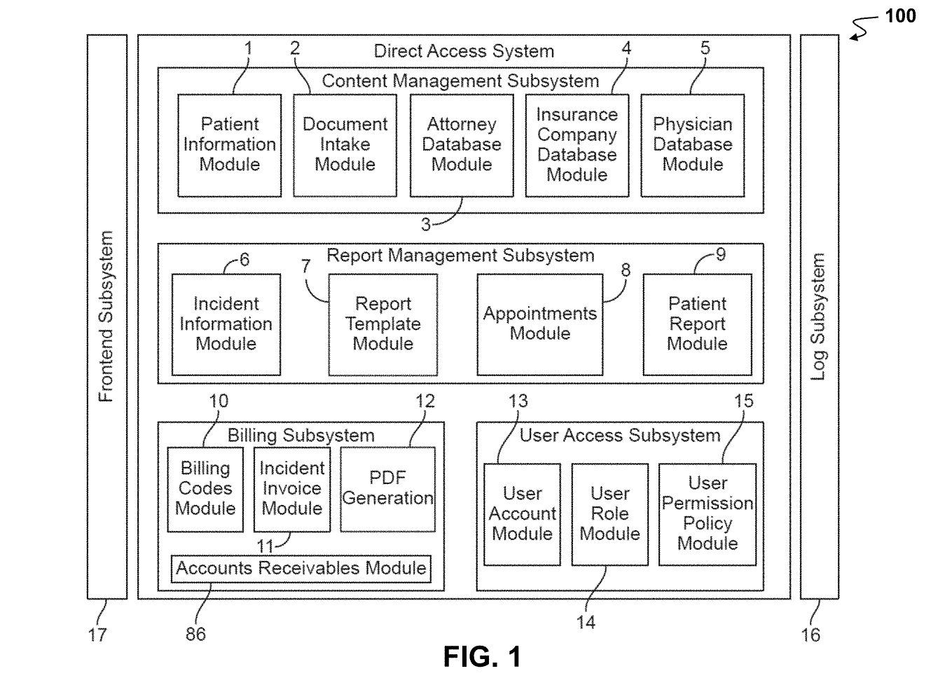

With reference to FIG. 1, the present embodiments include a conceptual block diagram illustration of a direct access system for patient medical records. In many embodiments, the direct access system can include a frontend subsystem 17 and a log subsystem 16. In a number of embodiments, the direct access subsystem may include a plurality of subsystems, with each subsystem containing various modules that can perform unique functions and communication with the rest of the direct access system. In certain embodiments, the subsystems in the direct access system may include a content management subsystem, a report management subsystem, a billing subsystem, and a user access subsystem. In further embodiments, a content management subsystem may include a patient information module 1, a document intake module 2, an attorney database module 3, an insurance company database module 4 and/or a physician database module 5. In still further embodiments, a report management subsystem can include at least an incident information module 6, a report template module 7, an appointments module 8, and/or a patient report module 9. In still yet further embodiments, a billing subsystem may include a billing codes module 10, an incident invoice module 11, a portable document format (PDF) generation module 12, and/or an accounts receivables module 86. In yet further embodiments, a user access subsystem may include a user account module 13, a user role module 14, and/or a user permission policy module 15.

While a variety of direct access systems for patient medical records are described above with reference to FIG. 1, the specific configurations, communication, and connections of the direct access systems for patient medical records are largely dependent upon the requirements of specific applications. For example, it can be appreciated by those skilled in the art that the exact types of modules and subsystems utilized by the direct access system can be adjusted and/or scaled based on the size, complexity, and/or processing needs of the users or clients. Additionally, the direct access systems for patient medical records can run entirely on a single consumer device or can be distributed over many devices, perhaps all utilized by the same user to provide a seamless experience at all levels of interaction. A more-detailed discussion of the subsystem modules is below.

### Patient Information Module

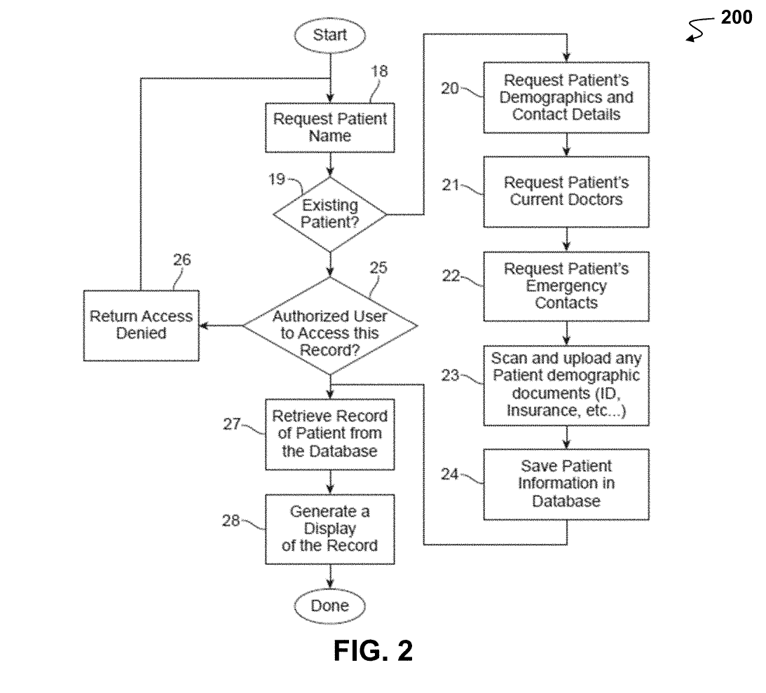

In various embodiments, the patient information module 1 can provide the functionality to create/edit/delete and view patient information (patient data). In most embodiments, all patient data is saved in a HIPPA compliant manner. In a variety of embodiments, patient information can be inputted into the patient information module 1 which can then save all of the patient data into a “patients” table in a database. FIG. 2 highlights an example process of an implementation that includes the utilization of the patient information module 1.

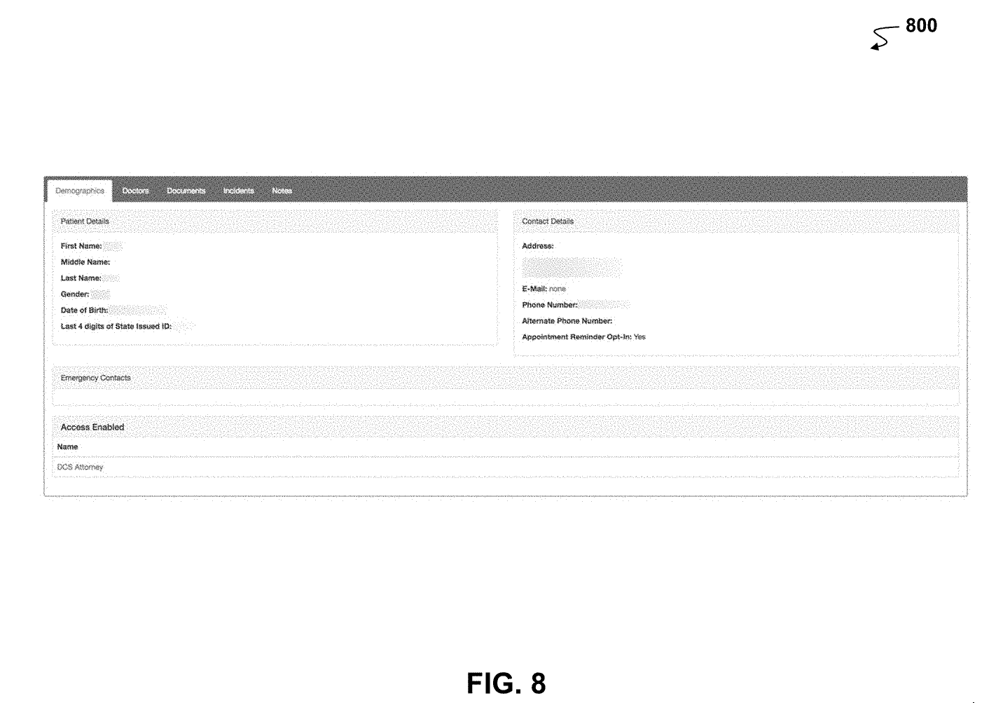

By way of example and not limitation, example screens of a direct access system for patient medical records utilizing a patient information module 1 are depicted in FIGS. 8-12. In additional embodiments, a patient's screen can show no patients initially by default. In still additional embodiments, input fields may include, but are not limited to, last name, first name, date of birth, MRN number, and/or phone number. In yet additional embodiments, a search for patients may yield a listing of various patients matching the search criteria. In still yet additional embodiments, the search results may allow for the sorting by MRN number and/or name. Additionally, certain embodiments may allow for the generation of an email with pre-filled in information when selecting an email link in the patient search results email field. In still yet additional embodiments, adding a new patient to the system can be accomplished with a new patient form 900 that can be added with the patient's information. In further embodiments, documents can be uploaded to the patient's records via a new patient document upload form 1000. In most embodiments, uploaded documents may be accessed on a documents tab in the patient's record. In more embodiments, any aspect of a patient's medical record may be edited by a user with sufficient user access privileges.

In the embodiment of FIG. 8, the screen 800 can include tabs for various sections, including, but not limited to:
1) Demographics—When first visiting a patient's profile, the default tab the user should see is the “Information” tab. This provides all the information the user inserted when creating the patient's profile or updating it.
2) Doctors—The “Doctors” tab lists all the doctors that were added to the patient's profile.
3) Documents—The “Documents” tab lists all the documents that have been uploaded to the patient's profile.
4) Incidents—The “Incidents” tab lists all of the incidents that have been added to the patient's profile.

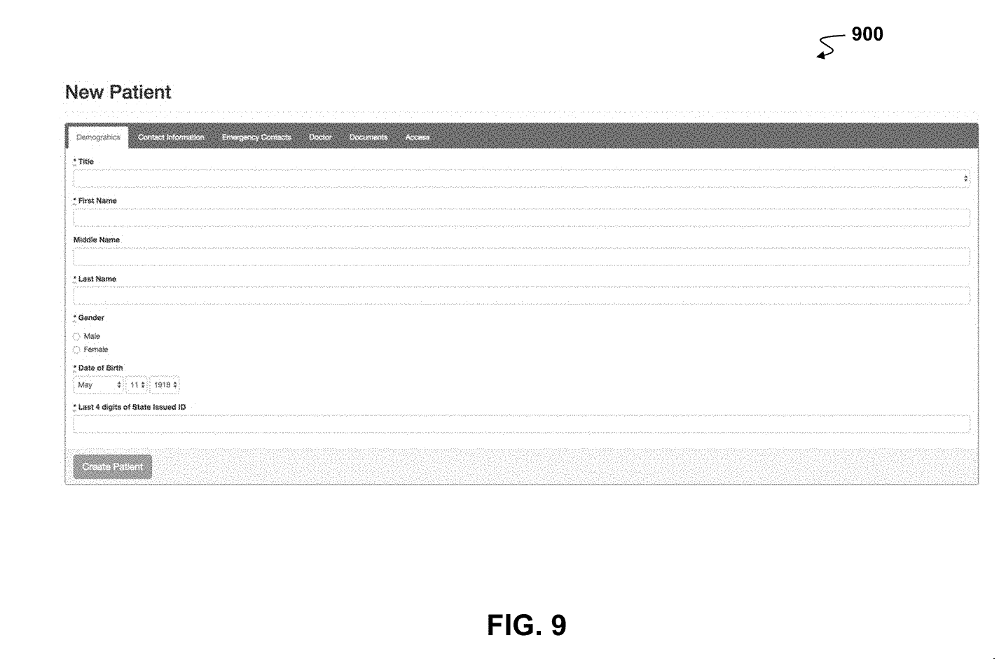

In the embodiment of FIG. 9, the screen can include fields for various items that can be filled out. In many embodiments, items with an asterisk (*) before the label is a required field and must be filled out. The fields can include, but not limited to:
1a) Title—Insert patient's salutation.
2a) First Name—Insert patient's first name into this field.
3a) Middle Name—If applicable, insert patient's middle name into this field.
4a) Last Name—Insert patient's last name into this field.
5a) Gender—Click one of the circles under the label “Gender” to identify whether the patient is a male or female.
6a) Date of Birth—To specify the patient's date of birth, simply click on the down arrow that represents the “month”, “day”, and “year” box and choose the month, day and year that corresponds with the date of birth provided by the patient.
7a) Last 4 Digits—Insert the last four digits of the patient's State Issued ID.

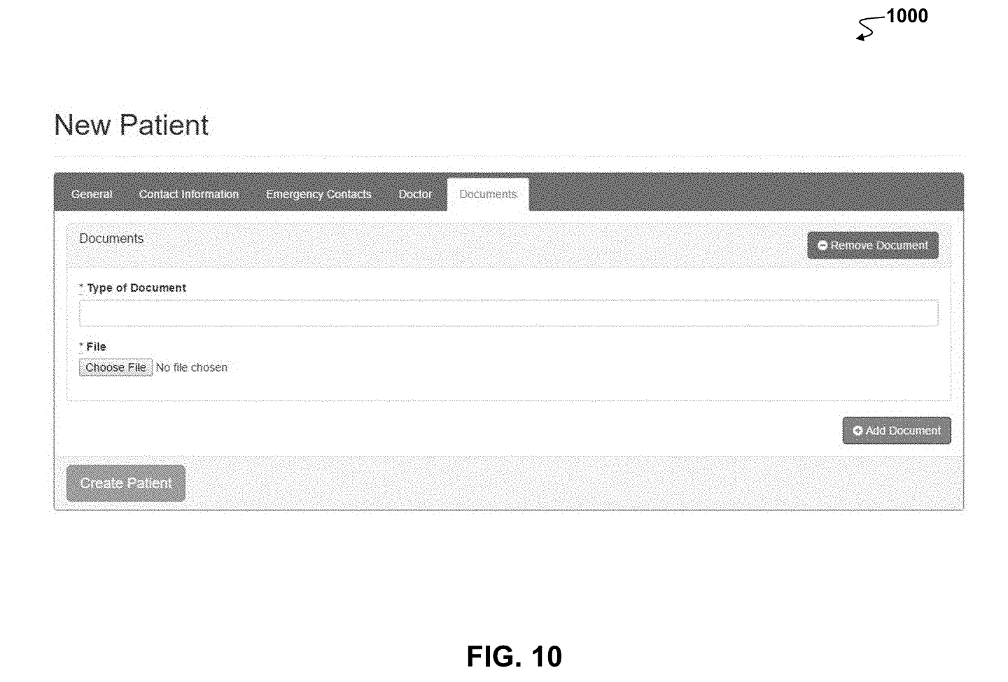

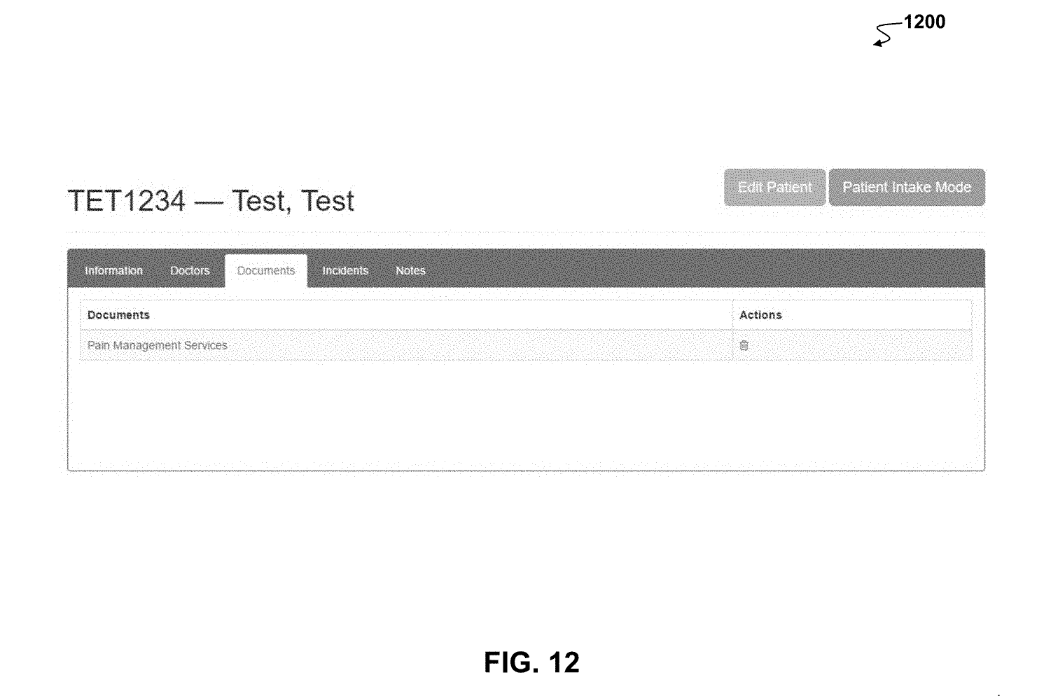

In the embodiment of FIG. 10, the screen can include fields for various items that can be filled out and/or buttons to select documents. In many embodiments, items with an asterisk (*) before the label is a required field and must be filled out. The fields can include, but not limited to:
2e) Type of Document—Insert a descriptive name for the document being uploaded into this field.
3e) File—Click on “Choose File” to upload a document directly from a computer or device.
4e) “Remove Document” Button—During any point throughout this process, click on the top right red button labeled “Remove Document” (FIG. 12) to remove the document from the patient's profile.
5e) “Add Document” Button—To upload additional documents, simply click the bottom right green button labeled “Add Document” (FIG. 12). This will bring up a new upload form.

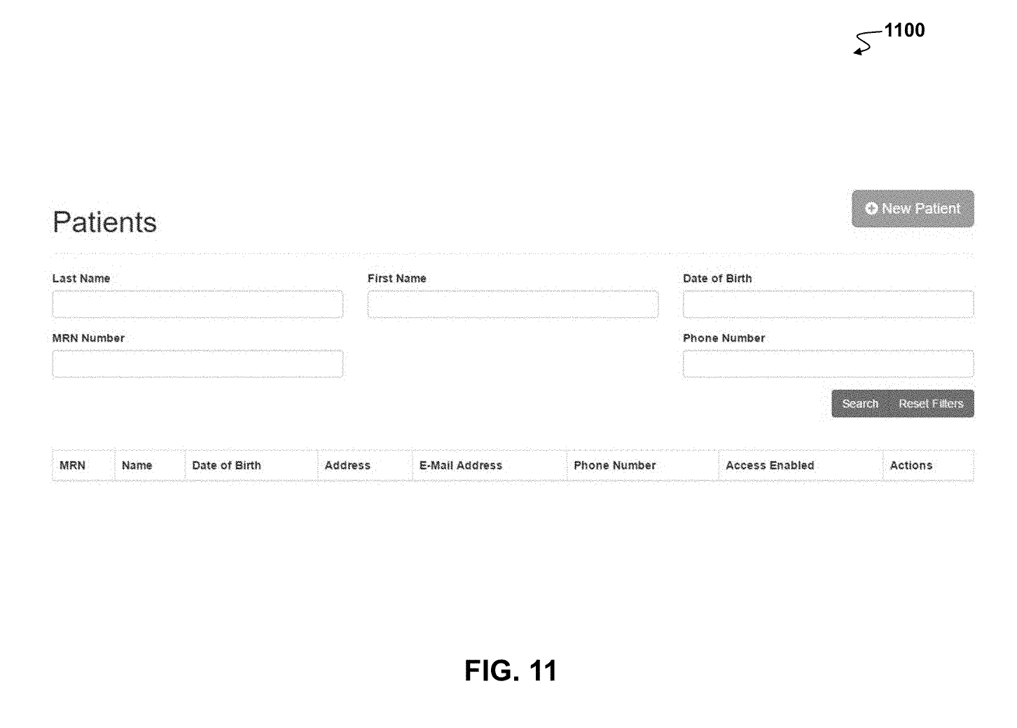

In the embodiment of FIG. 11, the screen can include fields for various items that can be filled out similar to FIG. 9, and/or buttons to adjust a search. The fields can include, but not limited to:
1) Patients—Attorneys/Physicians can view a list of patients assigned to them via the “Patients” tab.

In the embodiment of FIG. 12, the screen 1200 shows that all completed forms will be converted to a PDF file and uploaded directly to the patient's profile under the “Documents” tab.

### Document Intake Module

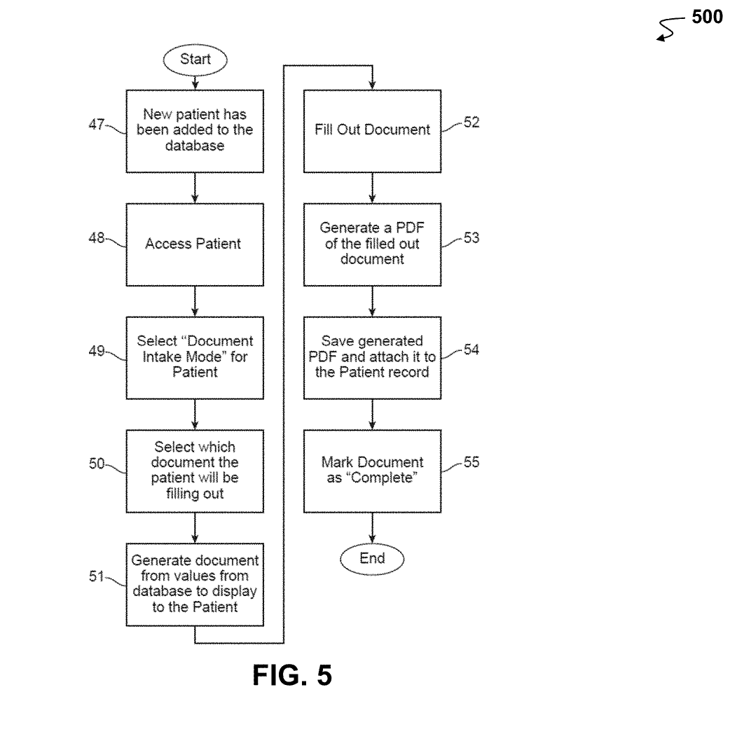

In many embodiments, the document intake module 2 may provide the functionality for patients to fill out any of a variety of patient intake forms via an access device such as, but not limited to, an iPad (or other similar portable computing device), a computer via PDF, or kiosk. In additional embodiments, the forms may be created and attached to the patient record in the system. FIG. 5 provides an example implementation of a process that can include utilizing the document intake module 2.

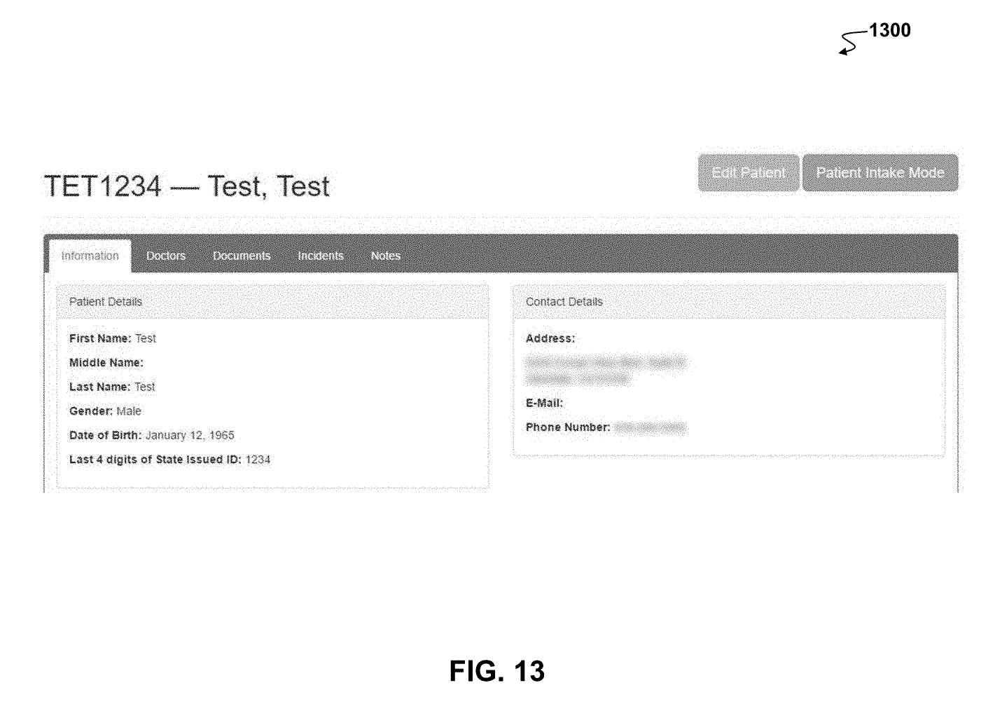

By way of example and not limitation, example screens of a direct access system for patient medical records utilizing a document intake module 2 are depicted in FIGS. 13-17. In further embodiments, a patient record presented in a search results screen may have a button that allows for a switch to patient intake mode. In still further embodiments, patient intake mode can begin by presenting the user with a list of forms 1400 that are required to be signed by the patient. By way of example and not limitation, an example form 1500 may be displayed to the user and presented to the patient for signing. In certain embodiments, the forms may be pre-filled out by pulling information from the patient record and entering information in the corresponding fields in the form. In a variety of embodiments, entry of the forms can be done electronically by the patient including, but not limited to a simplified date input 1600 which can enter a validated date into a form. In still more embodiments, the form may be presented to the patient for signing in a digital format by a variety of means including, but not limited to, a digital signature pad 1700 that may be signed by light pen and/or fingers. It would be obvious to those skilled in the art that any of these forms would be able to be edited and/or modified as needed based on new patient information.

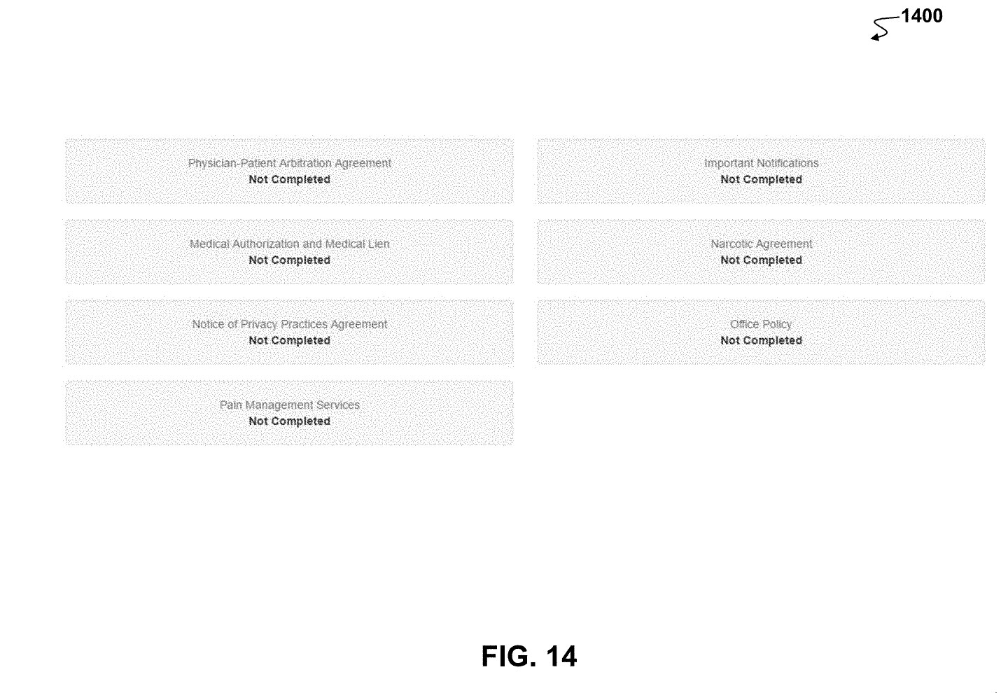

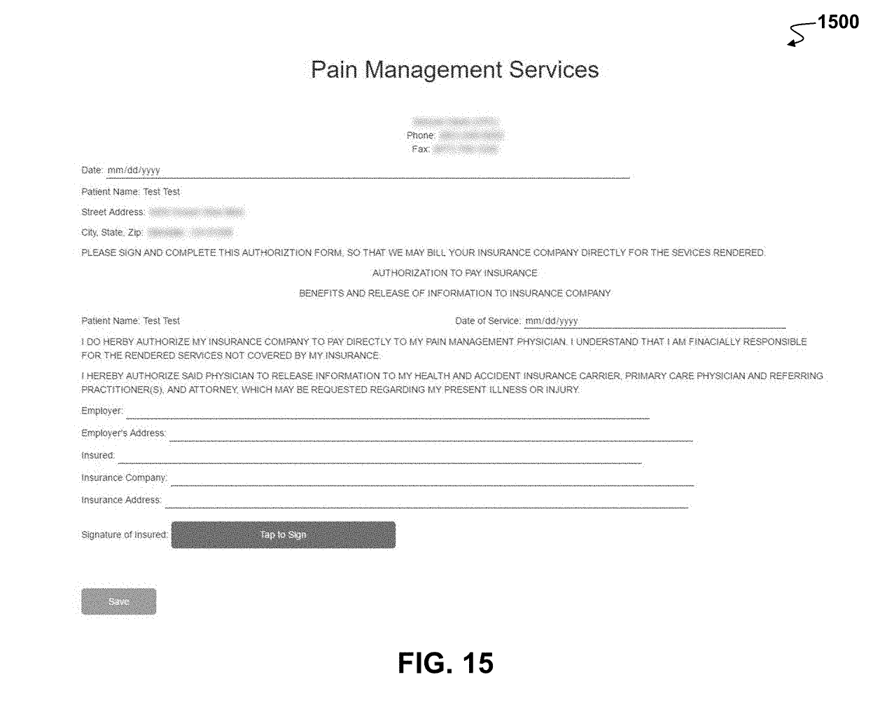

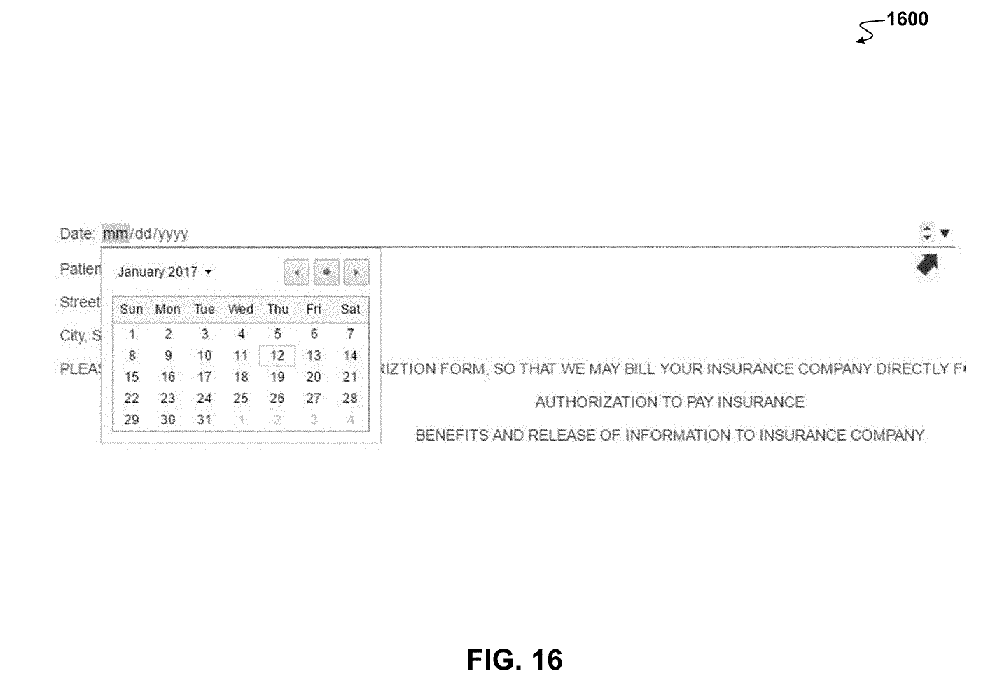

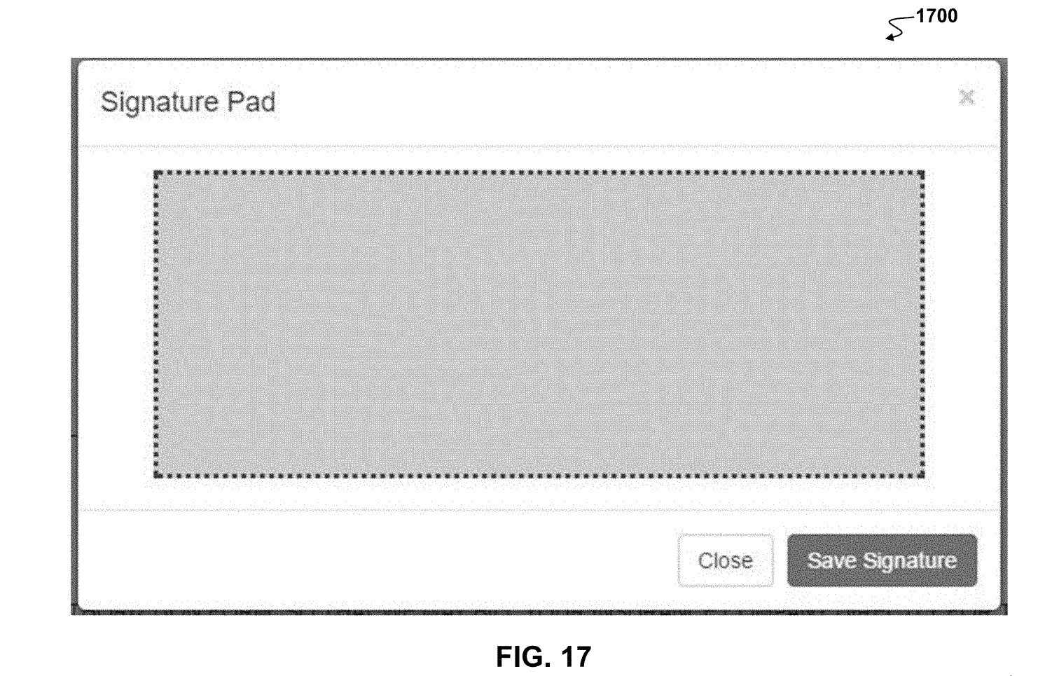

In the embodiment of FIG. 13, the screen 1300 can include tabs for various sections similar to FIG. 8 and information listed similar to FIG. 9. In many embodiments, once the user have searched and opened a specific patient's profile, the user can find a green button on the top right corner of the page labeled “Patient Intake Mode.” In a number of embodiments, clicking on “Patient Intake Mode” will direct the user to a page with different forms that need to be signed and completed by the patient such as, but not limited to, the forms in the embodiment of FIG. 14. To access one of the forms, a user may simply click on any of the form names and it will redirect them to that specific form. By way of example and not limitation, FIG. 15 is shown using “Pain Management Services” form. These forms can pull the patient's information, such as “Patient Name” for example, directly from their profile and insert it into the form automatically. All empty information lines that require the user to fill out specific information (Employer, Insurance Company, Signature, Etc.) can easily be accessed by clicking on any of the lines. Once clicked, the user will be able to type in any of the required information. In the embodiment of FIG. 16, inputting the date is made simple by inputting it manually by clicking on “mm,” “dd” or “yyyy” individually. The second option is to pick the date from a drop down calendar that is accessed by clicking the downward pointing arrow on the far right. In the embodiment depicted in FIG. 17, a signature pad is present. Every form may have a blue button labeled “Tap to Sign” which will require a signature to complete the form. By clicking on the blue button the user will bring up a signature pad. The signature pad is easy and simple to use. To sign simply use a finger (for iPads) or a mouse. Once all the required fields are completed, simply click the bottom left green button labeled “Save” to complete the form. Once a form is completed, patients will not be able to access that same form. This will be indicated with a check mark next inside the form box and labeled “Completed.”

### Attorney Database Module

In a number of embodiments, the attorney database module 3 may provide the functionality to create/edit/delete and view attorney information. In certain embodiments, attorneys can be invited to the system with a limited role to access their client's reports and bills. In further embodiments, an attorney screen can list of all law firms by default, which can contain all of the available information related to the specified law firms including, but not limited to, the case managers and incidents that they handle. In still further embodiments, attorneys can be added and edited as needed based on the information obtained and in fields that can be customized based on the required application.

### Insurance Agent Database Module

In various embodiments, the insurance agent database module 4 provides the functionality to create/edit/delete and view information about insurance companies and agents. In further embodiments, this information can be associated to an incident created for a patient. In additional embodiments, a list of all available insurance companies can be shown by default along with the available contact info. In still further embodiments, insurance companies can be added and edited as needed based on the information obtained and in fields that can be customized based on the required application.

### Physician Database Module

In a plurality of embodiments, the physician database module 5 can provide the functionality to create/edit/delete and view information about physicians. In still additional embodiments, physicians are associated to a patient's incident if a physician has referred a patient. In a number of embodiments, physicians (or practitioners) can be displayed to indicate their profession/specialty, and the types of incidents they handle. In additional embodiments, physicians can be added and edited as needed based on the information obtained and in fields that can be customized based on the required application.

### Incident Information Module

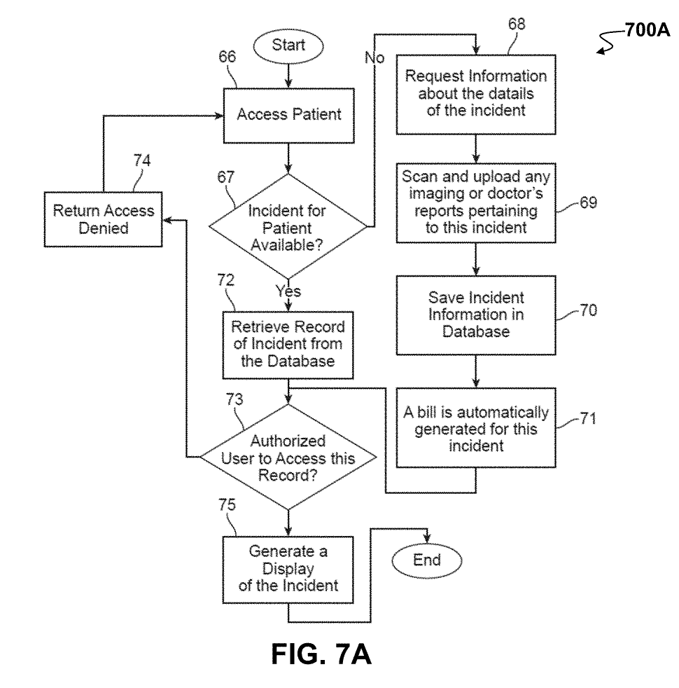

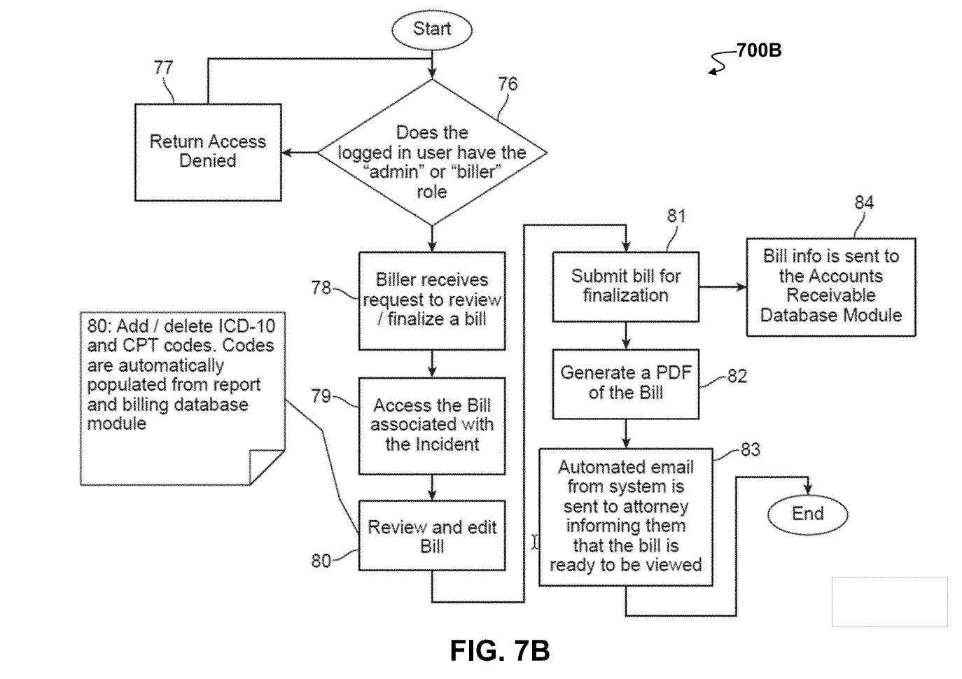

In more embodiments, the incident information module 6 may provide the functionality to create/edit/delete and view information about incidents. Generally, incidents can be classified as a collection of data related to a patient's case and may contain all medical documents processed related to the specific information and/or event as well as any attorney, insurance, and/or physician details. In further embodiments, incidents information can include the collection of reports and bills that are associated with a patient's particular accident and/or incident. In certain embodiments, appointments can also be created and associated with incidents. FIG. 7A depicts an example process that can provide an example implementation of the incident information module 6.

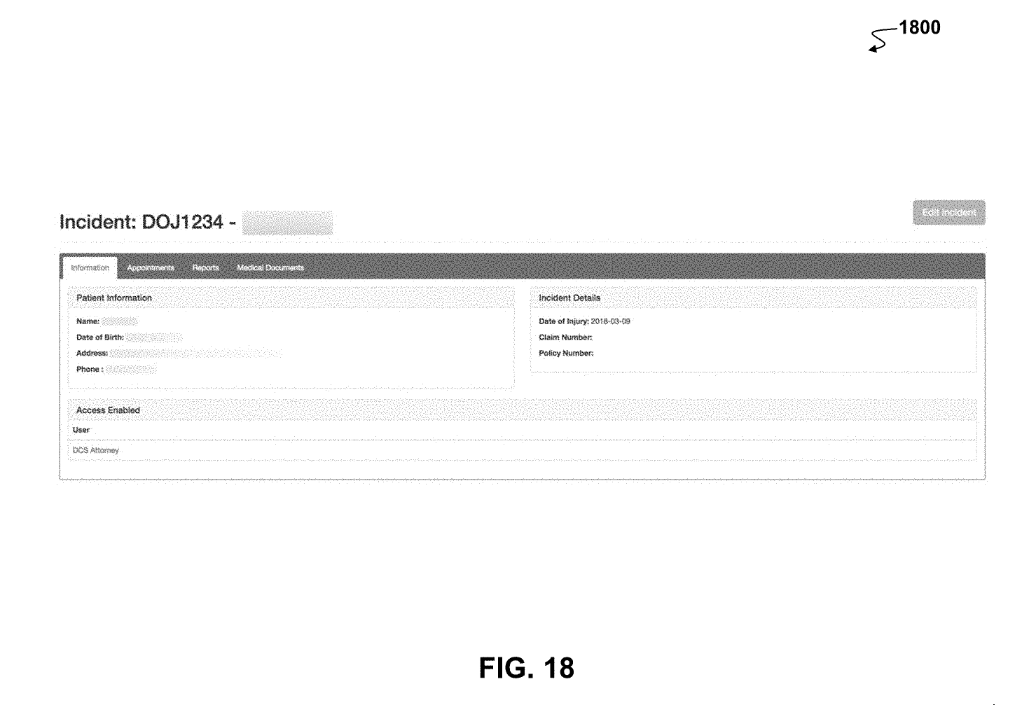

By way of example and not limitation, example screens of a direct access system for patient medical records utilizing an incident information module 6 are depicted in FIGS. 18-21. In more embodiments, an incident may be viewed by a user with sufficient user privileges. Additionally, in still more embodiments, incidents can be added and edited as needed based on the information obtained and in fields that can be customized based on the required application. In yet more embodiments, an incident may include an initial consultation that may be added along with any necessary documents. In still yet more embodiments, an operative report may also be added to an incident which may include, but is not limited to, caudal epidurals, trigger point injections, and/or other pain management. In other more embodiments, the incident information may be utilized to generate a finalized report with pre-filled in fields and/or other text.

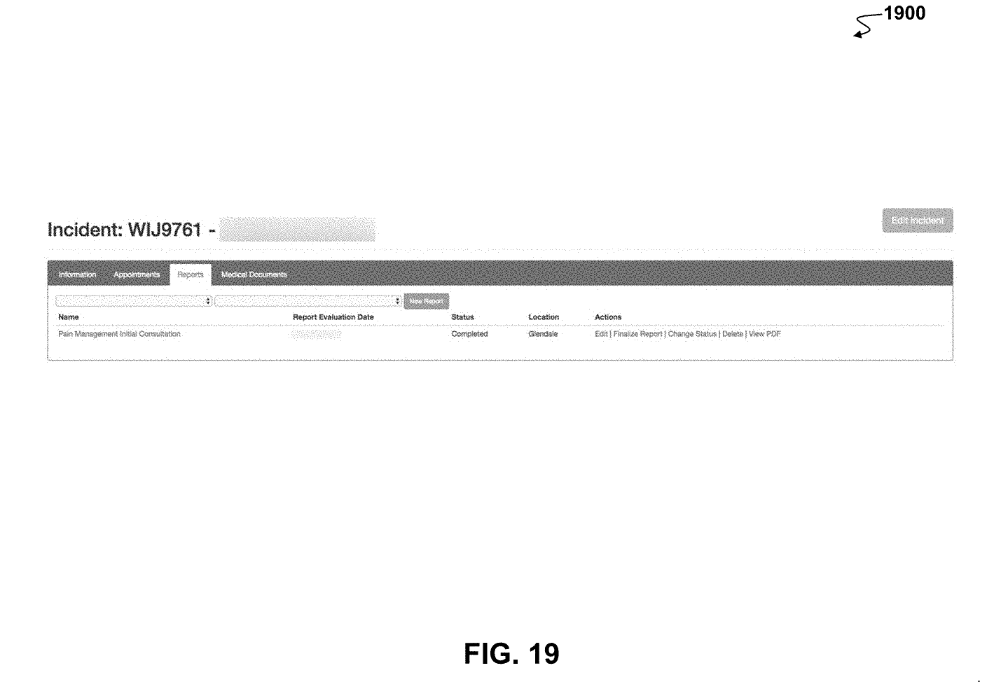

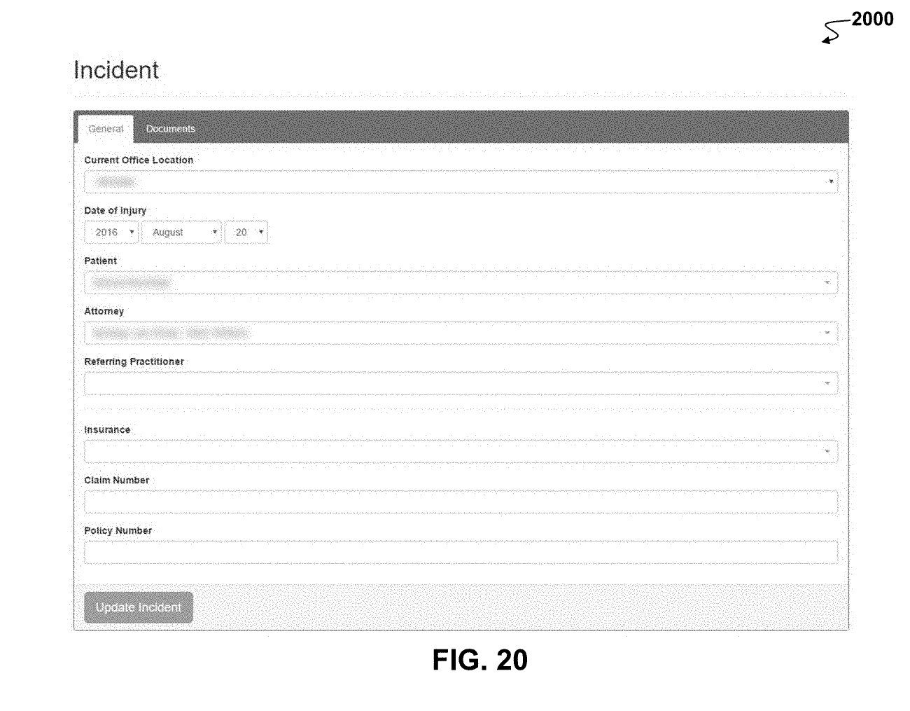

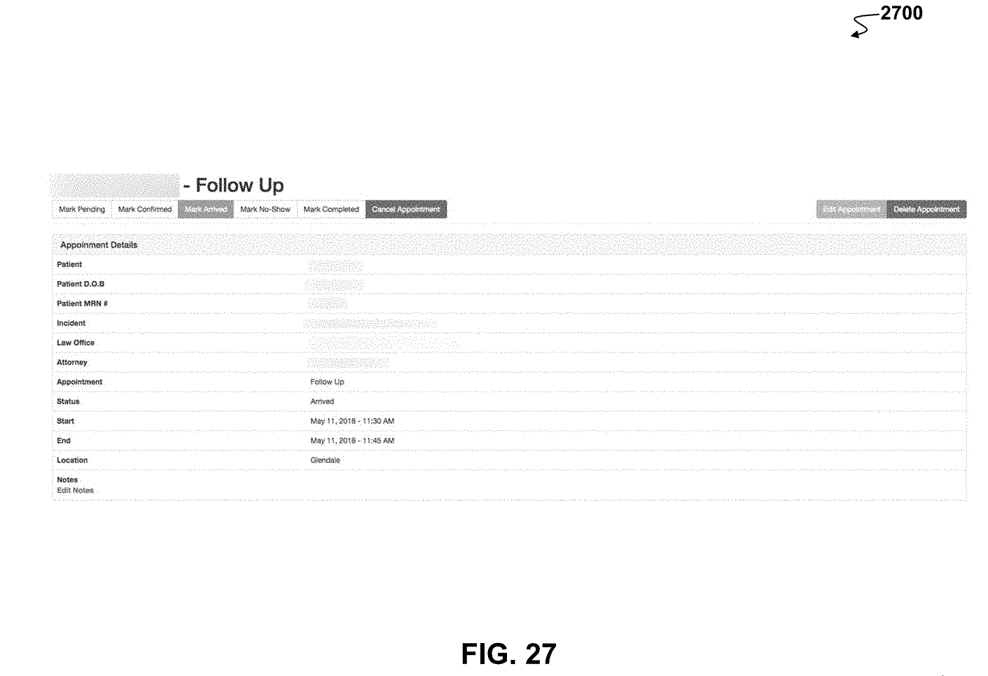

In the embodiment depicted in FIG. 18, there are two simple ways to view an incident on an incident information screen 1800. The first option can be to click on a patient's name on the search field. The second option is to click on the date of injury corresponding with the patient's incident the user wish to view. Clicking either one of these options will bring up the page displayed in FIG. 18. The “Reports” tab screen 1900, as depicted in FIG. 19 will display all the incident reports corresponding with a specific client. Here the user can create follow ups, finalize reports, etc. The user can also view the Incident Report by clicking on the “Name” of the report. In the embodiment shown in FIG. 20, an incident report screen 2000 may include a number of fields which can include, but are not limited to:
1a) Current Office Location—This field has a simple drop down menu which allows the user to select the current office location from a variety of different locations.
2a) Date of Injury—Input the date of injury provided by a patient into this field.
3a) Patient—This field has a simple drop down menu which allows the user to select a specific patient from a list of all patients. The user also has the option of searching for a specific patient by name once the user has accessed the drop down menu.
4a) Attorney—This field has a simple drop down menu which allows the user to select a specific attorney from a variety of different attorneys. The user also has the option of searching for a specific attorney by name once the user has accessed the drop down menu.
5a) Referring Practitioner—This field has a simple drop down menu which allows the user to select a practitioner from who the user was referred from. The user also has the option of searching for a specific practitioner by name once the user has accessed the drop down menu.
6a) Insurance—Unlike the other fields, the drop down menu for this field will give the user the option to type in the patient's insurance. The user will be given options once the user start typing with results that best match the patient's input.
7a) Claim Number—Input a claim number into this field that best corresponds with a patient's claim number.
8a) Policy Number—Input a policy number into this field that best corresponds with a patient's policy number.
9a) “Update Incident” Button—Once the user have completed inputting all the information into these fields, simply click the green button on the bottom labeled “Update Incident” (FIG. 27) to finish editing.

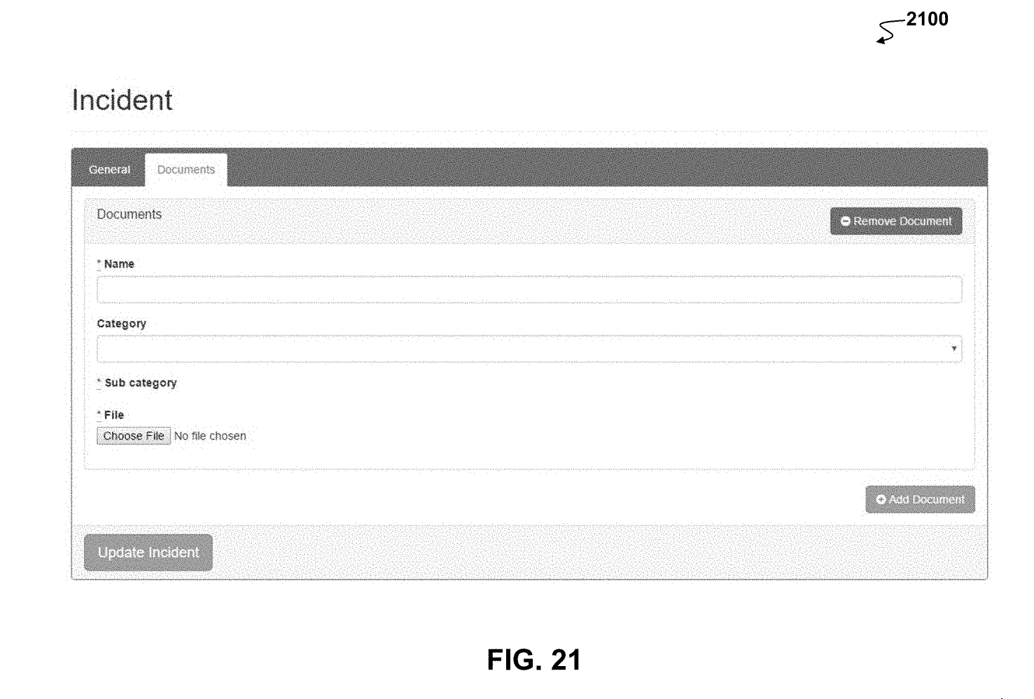

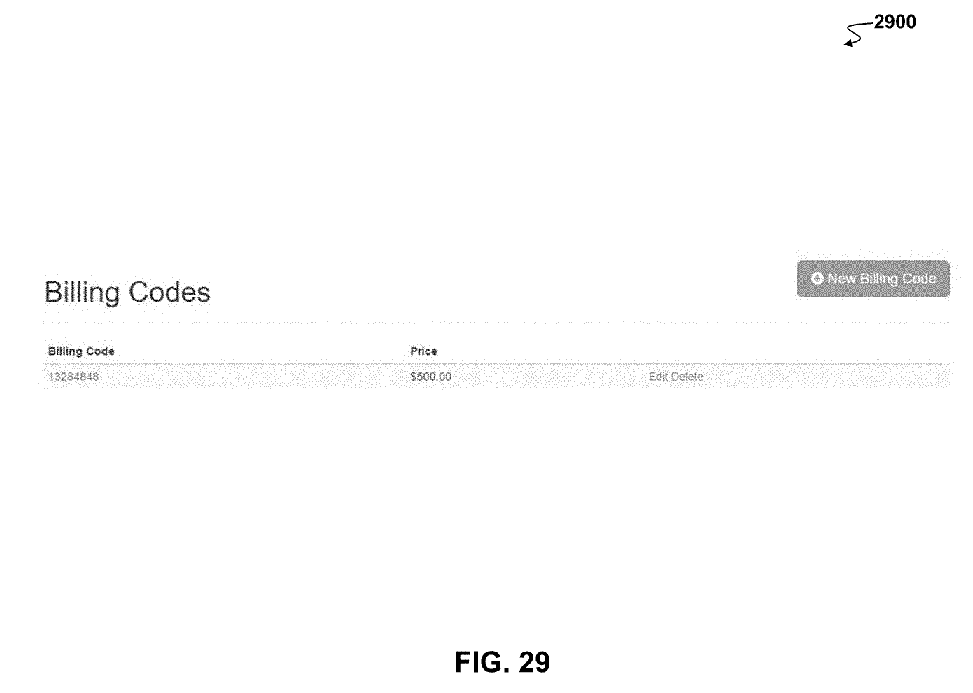

The “Documents” tab screen 2100 is where all the documents that have been uploaded regarding that specific incident and is depicted in FIG. 21. The user can add and remove documents directly from this section. The fields that may be present in the documents tab include, but are not limited to:
2b) Name—Input name of the document the user wish to upload into this field.
3b) Category—This field has a simple drop down menu with all the available categories in which the user can choose from.
4b) Sub Category—Once the user has selected a category, an option to select a sub category will be displayed. Simply choose which sub category best corresponds with the document being uploaded.
5b) File—Clicking “Choose File” will pop up a window allowing the user to upload a document directly from a computer or device.
6b) “Remove Document” Button—During any point throughout this process, click on the top right red button labeled “Remove Document” (FIG. 29) to remove the document from the incident.
7b) “Update Incident” Button—Once the user have completed inputting all the information into these fields, simply click the green button on the bottom labeled “Update Incident” (FIG. 29 above).

### Report Template Module

In many embodiments, the report template module 7 can provide the functionality to define report templates. In still further embodiments, report templates typically are the collection of“questions” that the doctor fills out when creating a report.

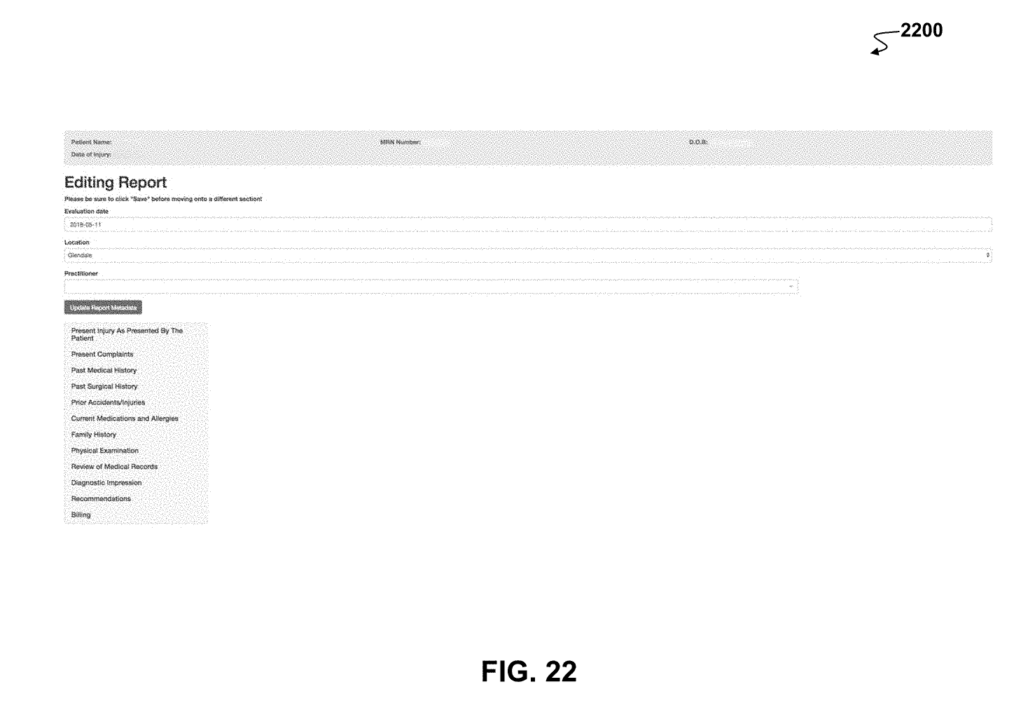

By way of example and not limitation, example screens of a direct access system for patient medical records utilizing a report template module 7 are depicted in FIGS. 22-24. The embodiment shown in FIG. 22 is an editing report screen 2200 with fields including, but not limited to:
2a) Evaluation Date—Begin filling out the form by inserting an evaluation date for the incident. There is no need to type the date as there is a calendar pop-up making it extremely simple to input data into this field.
2b) Location—This field has a simple drop down menu which allows the user to select the current office location from a variety of different locations.
2c) Practitioner—This field has text input which allows the user to input a practitioner.
3a) Patient Information—The rest of the form is made extremely easy and simple. There are general tabs to insert more information about the patient's complaints, medical history, family history, etc. Each section has a checklist in which the user can check all that applies to the patient.
4a) Saving the Report—After the user has completed all the fields that apply, simply click on the bottom right blue button labeled “Save Report.”

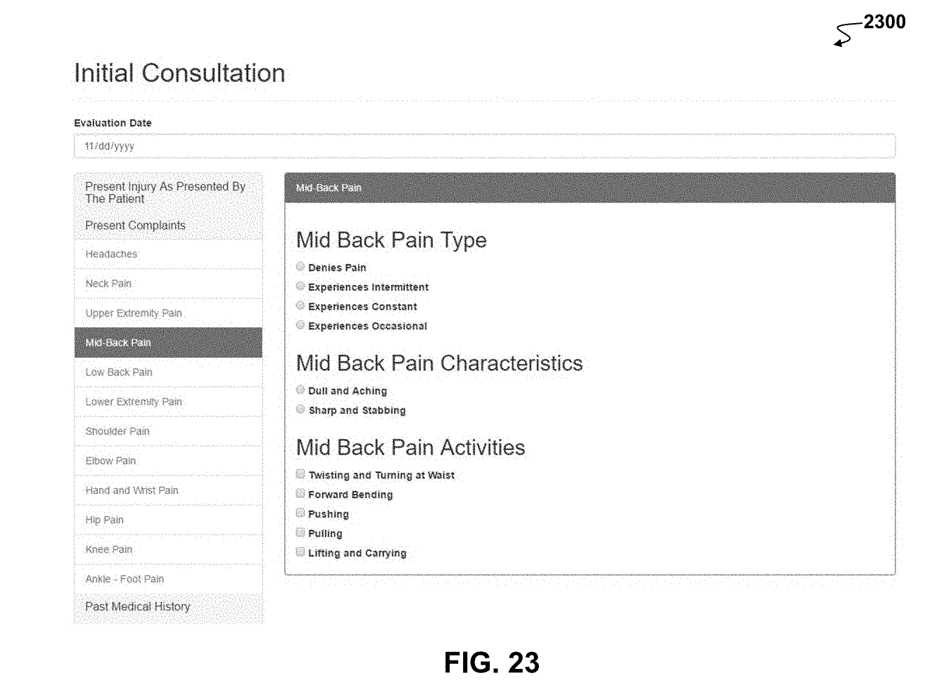

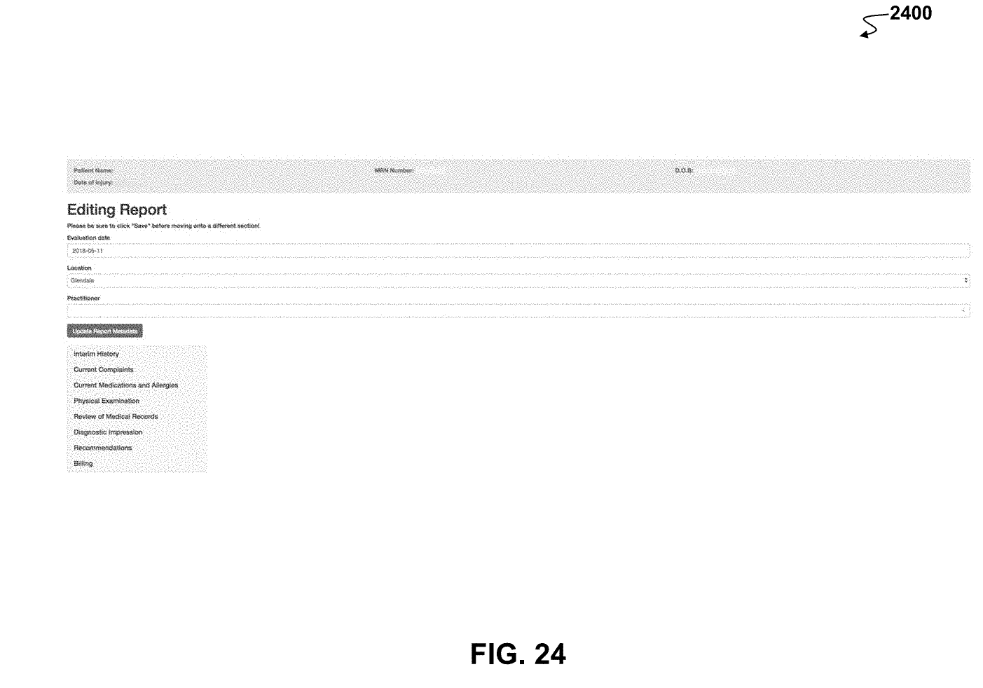

An initial consultation screen 2300 is depicted in FIG. 23. The editing report screen 2400 embodiment depicted in FIG. 24 has a series of fields similar to FIG. 22 which includes, but is not limited, to:
2d) Evaluation Date—Begin filling out the form by inserting an evaluation date for the incident. There is no need to type the date as there is a calendar pop-up making it extremely simple to input data into this field.
3d) Operative Report—Much like the “Initial Consultation” form, there are general tabs on the left that open up a form with checklists which make it simple and easy for the user to fill out. Check any of the options that apply to a patient.
4d) Saving the Report—After the user has completed all the fields that apply, the user may simply click on the bottom right blue button labeled “Save Report.”

### Appointments Module

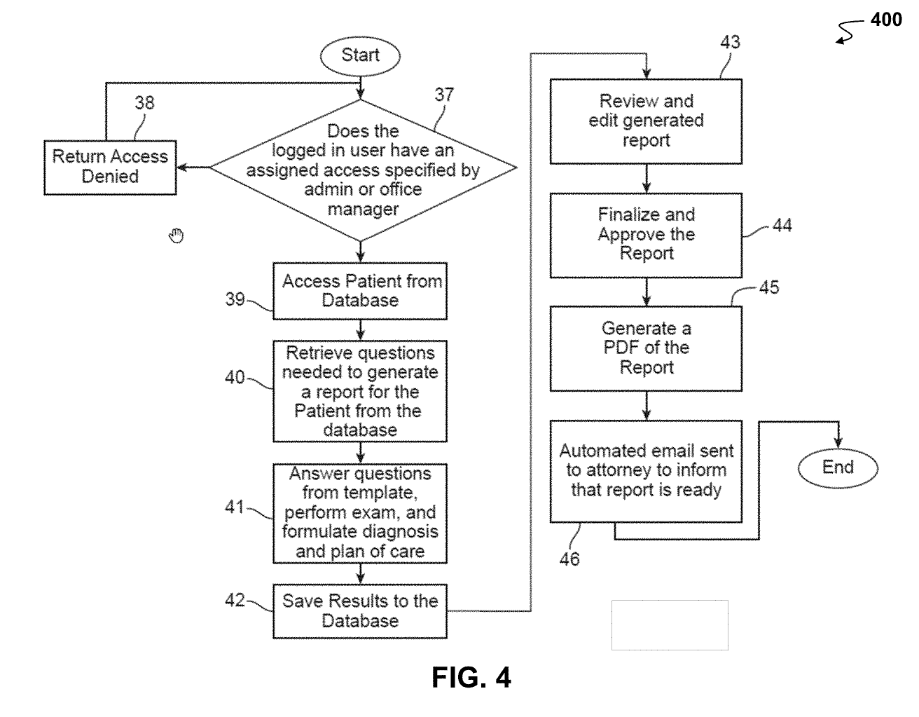

In a variety of embodiments, the appointments module 8 may provide the functionality to create, edit, and/or delete appointments. In additional embodiments, appointments can be either created by a front office staff, an assigned attorney, or an assigned practitioner for their client. In still additional embodiments, appointments have statuses that can be assigned to them which must generally be confirmed by the doctor's office. In yet additional embodiments, an automated email can be sent out by the system to an outside user once the appointment is confirmed by the office staff. FIG. 4 illustrates an example process that can provide an implementation of the Appointments Module 8.

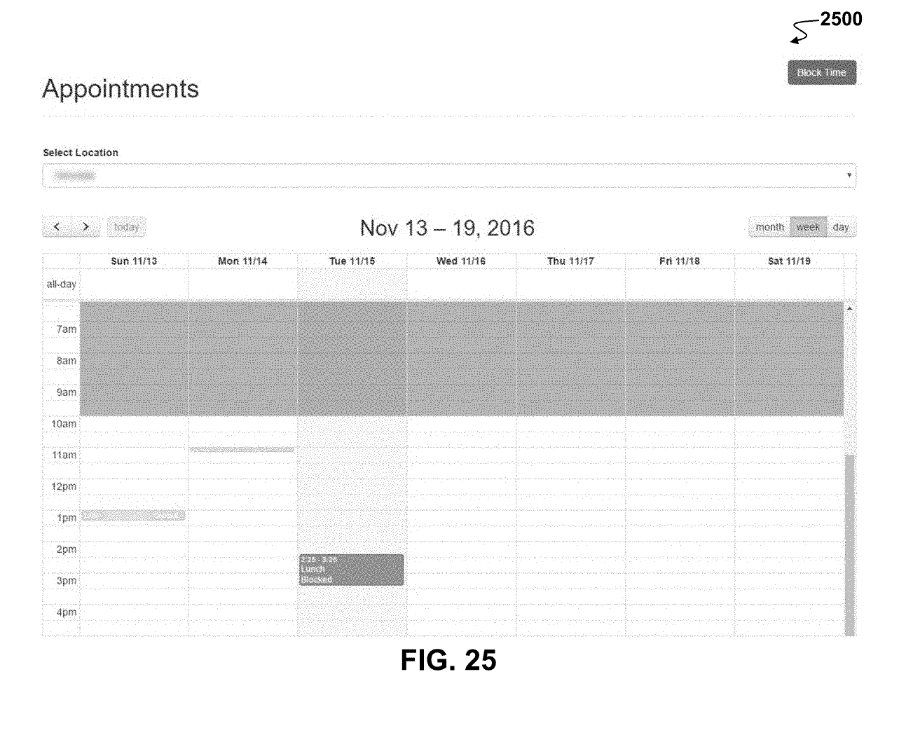

By way of example and not limitation, example screens of a direct access system for patient medical records utilizing an appointments module 8 are depicted in FIGS. 25-28. In further embodiments, appointments can be utilized to view either calendar or report views. In still further embodiments, a calendar view 2500 may be utilized to create, edit, or delete appointments and/or to block time. In yet further embodiments, time may be blocked off by entry of information into a block time screen 2600. In still yet further embodiments, clicking on an appointment in the calendar view can open up an appointment details screen 2700 where the details of an appointment can be visible to a user with sufficient privileges. In additional embodiments, outside users including, but not limited to, attorneys may be able to see an appointment but may not be able to utilize or see certain features like cancelling or deleting appointments. In still additional embodiments, an appointment report screen 2800 may be generated to display a history of appointments made, with information related to each appointment such as, but not limited to, status, type, start and end times, appointment creator, and/or office location.

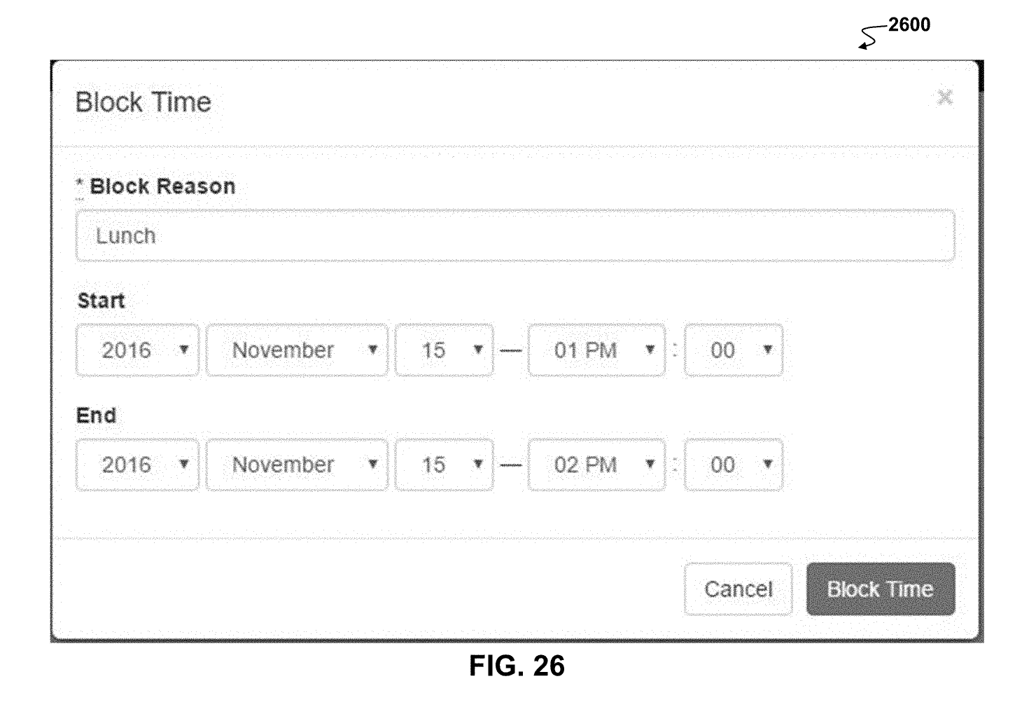

In the embodiment depicted in FIG. 25, a calendar is shown. To begin viewing appointments, a user must first select a location. There are three options for viewing the calendar; monthly, weekly or by day. The red button on the top right corner allows for office representatives of a specific location to “block” certain times that the office will not be available for appointments. The window embodiment depicted in FIG. 26 shows a block time window and allows a user to input a reason for the block time (This can be a required field), and to input a time frame from which the office will be unavailable. In many embodiments, Attorneys can view appointment times and block times, however, they are unable to see the reason for the appointment or block time. Instead, they are labeled as “Not Available.” In the screen shown in FIG. 27, clicking on any of the appointments in FIG. 25 will redirect the user to this screen and allows the user to mark the appointment accordingly, cancel the appointment or delete the appointment. The appointment details screen in FIG. 27 has a series of fields including, but not limited to:
3) Block Time—The red button on the top right corner allows for office representatives of a specific location to “block” certain times that the office will not be available for appointments (Input a reason for the block time (This can be a required field). Also, input a time frame from which the office will be unavailable.
4) Mark Confirmed/Pending—Mark a pending appointment as “confirmed” or mark a confirmed appointment as “pending.”
5) Mark Arrived—Once the patient has arrived to the office, mark the appointment as “arrived.”
6) Mark No-Show—If the patient did not show up to their appointment and gave no notification ahead of time, mark the appointment as “no-show.”
7) Mark Completed—Once the appointment is done and the patient has left the office, mark the appointment as “completed.”
8) Cancel Appointment—If a patient notifies that they are unable to make it to their appointment, click the red “Cancel Appointment” button.
9) Delete Appointment—Unlike “Cancel Appointment,” deleting the appointment will erase it from the system.
10) Calendar Legend—
   Gray: Blocked
   Green: Confirmed
   Lime Green: Pending
   Red: Canceled
   Dark Green: Arrived
   Black: No Show
   Yellow: Requested
   Brown: Declined
   Blue: Completed

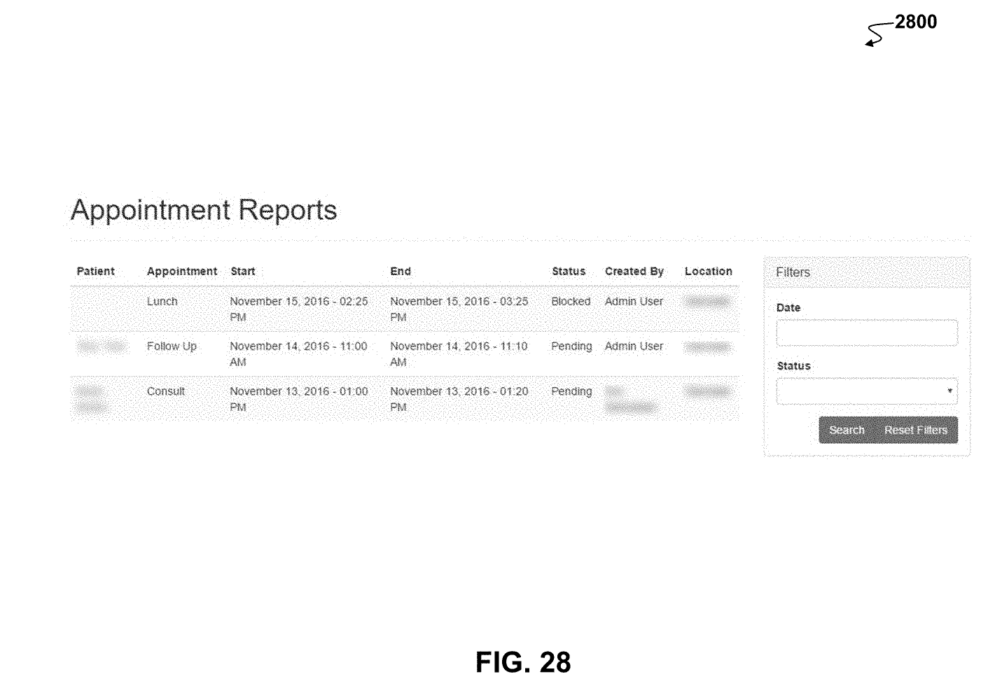

Note that attorneys are also able to access the “Appointment Details” page; however, they are unable to mark, cancel or delete the appointments. The appointment reports screen embodiment shown in FIG. 28 displays a history of appointments made. This table provides brief information about the appointment such as status, appointment type, start/end of scheduled appointment, appointment status, who created the appointment and the location of the office the appointment is scheduled for. Uses can filter the appointment reports by date or by status. Input both date and status or one of the two and click “Search” to filter the table. Clicking “Reset Filters” will clear both date and status and also reset the table to show all the results.

### Patient Report Module

In more embodiments, the patient report module 9 can provide the functionality to create, edit, delete, finalize, and/or view reports. In still more embodiments, reports can be generated by a doctor by answering a series of predefined “questions”, entering examination values, choosing diagnoses and/or treatment recommendations that are created in a report template module 7. In even more embodiments, these reports are then reviewed and “signed off” by a doctor. In yet more embodiments, completed reports may be available to be downloaded by the users assigned to the incident. FIG. 5 illustrates an example process that can provide an implementation of the patient report module 9.

### Billing Codes Module

In a variety of embodiments, the billing code module 10 can provide the functionality to define the various billing codes that will be used on a potential bill. In certain embodiments, the billing codes may have a price associated with them.

By way of example and not limitation, example screens of a direct access system for patient medical records utilizing a billing codes module 10 are depicted in FIG. 29. In more embodiments, when an incident is created, a bill can automatically be created as well, which can be viewed in a list of bills. As those skilled in the art will recognize, billing codes and associated bills may change and/or be edited or deleted as new information is presented. In still more embodiments, a billing codes screen 2900 may be generated that lists all of the available bills for a patient. In yet more embodiments, bills may be presented as a billing code with an associated price (if available). In the bill codes screen embodiment of FIG. 29, billing codes can be found by clicking “Settings” to access the dropdown menu, followed by clicking on “Billing Codes.”

### Incident Invoice Module

In a plurality of embodiments, the incident invoice module 11 can enable associating a bill to each incident. In certain embodiments, the bill can automatically be updated based on reports created by a doctor. In further embodiments, once a report is finalized or “signed off”, an automated email can be sent out by the system to the biller informing of a bill being ready for viewing, editing, and/or finalizing. In still further embodiments, the bill can be reviewed by the biller and upon completion may be converted to a PDF for an attorney to download.

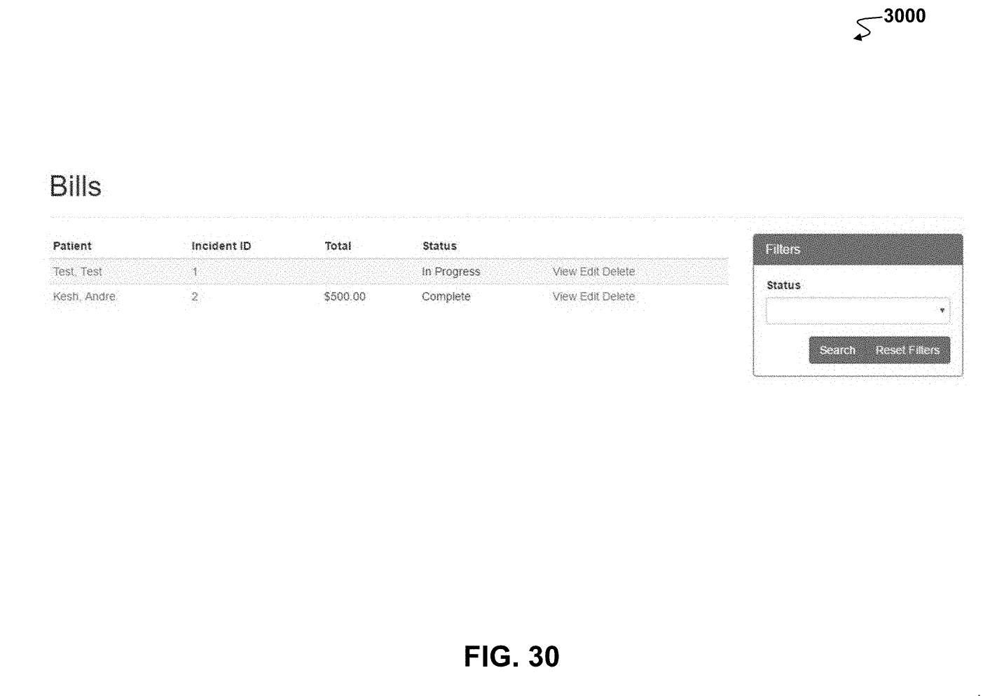

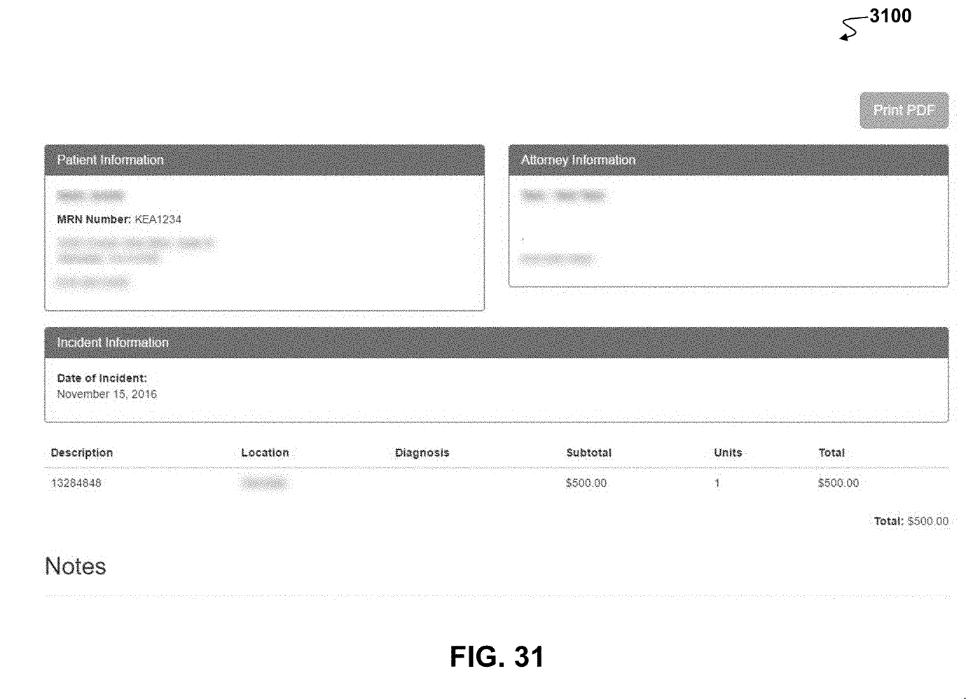

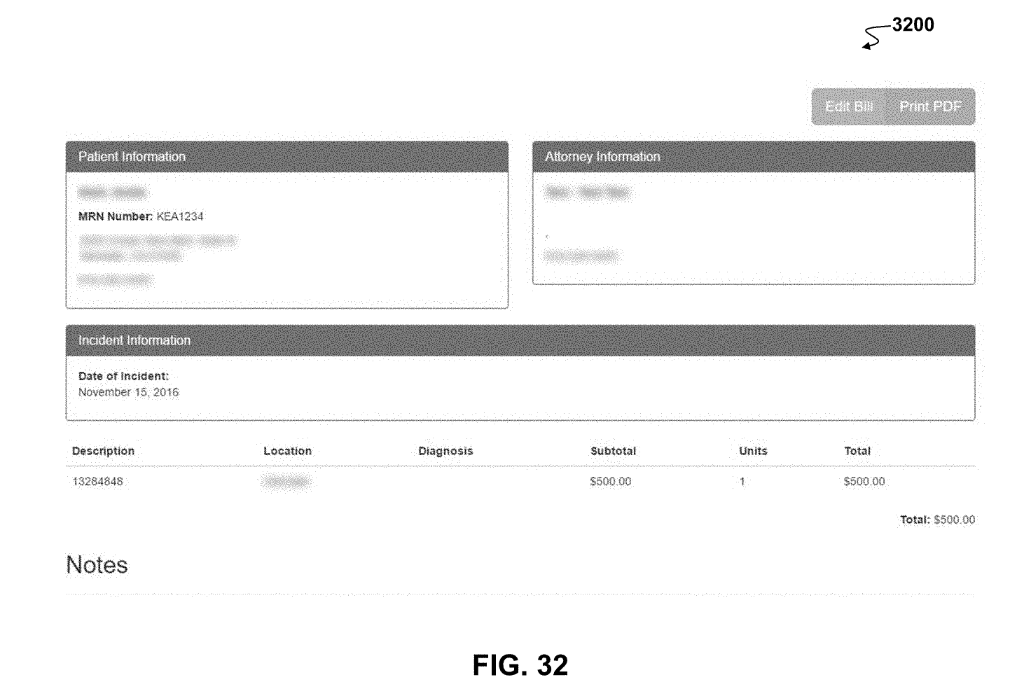

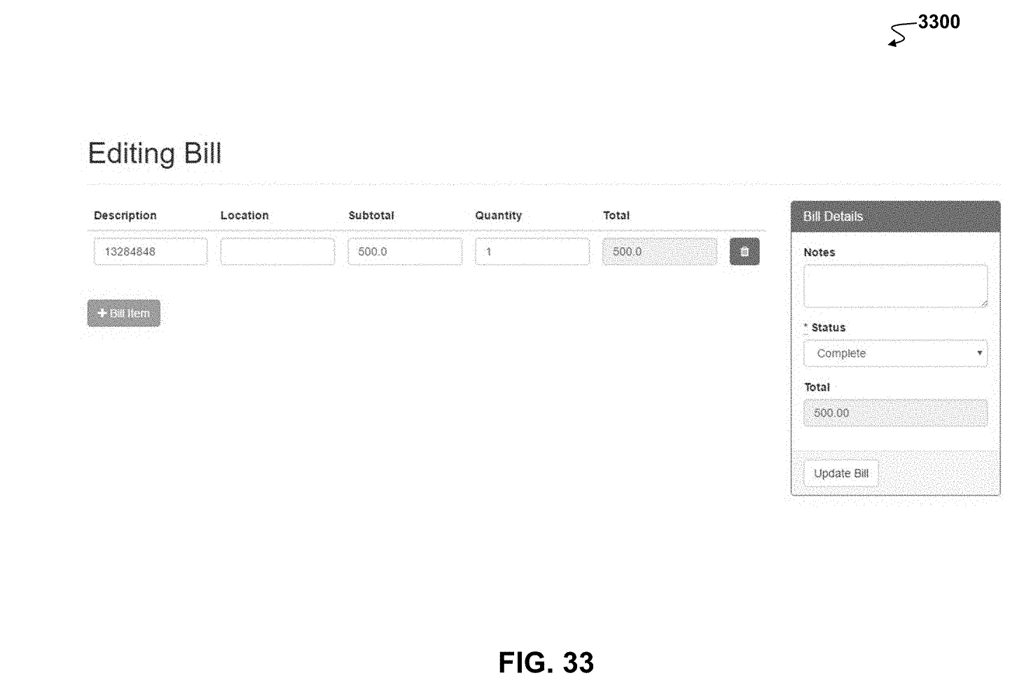

By way of example and not limitation, example screens of a direct access system for patient medical records utilizing an incident invoice module 11 are depicted in FIGS. 30-33. In a number of embodiments, the incident invoice module may work in a similar manner as the billing codes module 10, allowing for all of the same displays, and creation and editing capabilities. In the Bills screen 3000 embodiment depicted in FIG. 30, clicking on “View” or the patient's name will redirect the user to the specified billing page displayed in FIG. 31. The user can view a list of bills by clicking the tab called “Bills,” this will redirect the user the page displayed in FIG. 30. Also, in many embodiments, clicking on the top right blue button labeled “Print PDF” as seen in the patient information screen 3100 in FIG. 31, will allow the user to print a pdf with all the billing information for that specific bill. In the patient information screen 3200 embodiment shown in FIG. 32, Admin and Office Manager Users may have the ability to edit a bill. When viewing the list of bills displayed in FIG. 30, clicking on the patient name will display a slightly different billing information page than displayed in FIG. 31. In many embodiments, the only difference for Office Managers/Admins is that they will see an orange button labeled “Edit Bill” on the top right corner of FIG. 32. Clicking this will redirect the user to the screen 3300 displayed in FIG. 33. In certain embodiments, the editing bill screen embodiment in FIG. 33 can include many fields including, but not limited to:
1a) Add a Bill Item—Clicking on the bottom left green button labeled “+Bill Item” will add a new row for the user to input new billing item information.
1b) Delete a Bill Item—Clicking the red button labeled with a trashcan Icon will delete the billing item from the bill.
1c) Notes—Input any notes for the bill.
1d) Status—This is a required field. Input whether the status is In Progress, Pending or Complete.
1e) Description—Input the billing code into this field.
1f) Location—Input the specific location that corresponds with the bill item.
1g) Subtotal—Input the subtotal of the bill item.
1h) Quantity—Input the quantity of the bill item.
1i) Update Bill—Clicking the white button labeled “Update Bill” under “Bill Details” will update all the changes made to the bill.

In a number of embodiments, when a report is created, it can automatically take into account the diagnostic impression and the time needed to review the records. The user can manually add a line item (There is a checkbox to make things much simpler) for the diagnostic impression. Also note, a new line item is automatically created for the time needed to review the records.

### PDF Generation Module

In still yet further embodiments, a PDF generation module 12 may provide the functionality to convert a bill into a downloadable PDF file. Those skilled in the art will recognize that the PDF generation module 12 may easily be replaced and/or supplemented with a similar generation module that can output the necessary data in a data format that is suitable based on the needs of the user application.

### Accounts Receivables Module

In more embodiments, the accounts receivables module 86 may provide the functionality that allows a Biller, Admin, and/or Office Manager to generate a report showing the money owed to the practice. In still more embodiments, the value can be populated from the incident bill which may have been generated by the system prior. In even more embodiments, the information can be organized by name, medical record number (MRN), incident, total bill, amount paid, amount adjusted, and/or case status.

### User Account Module

In a number of embodiments, the user account module 13 can provide the functionality that allows users to be created, edited and/or deleted. In more embodiments, the user account module can also contain functionality that can allow for the user to login or logout.

### User Role Module

In a variety of embodiments, a user role module 14 may provide functionality to define roles that are assigned to a user.

### User Permission Policy Module

In a plurality of embodiments, a user permission policy module 15 can provide the functionality to define an authorization to different modules of the system based on the logged in user's role.

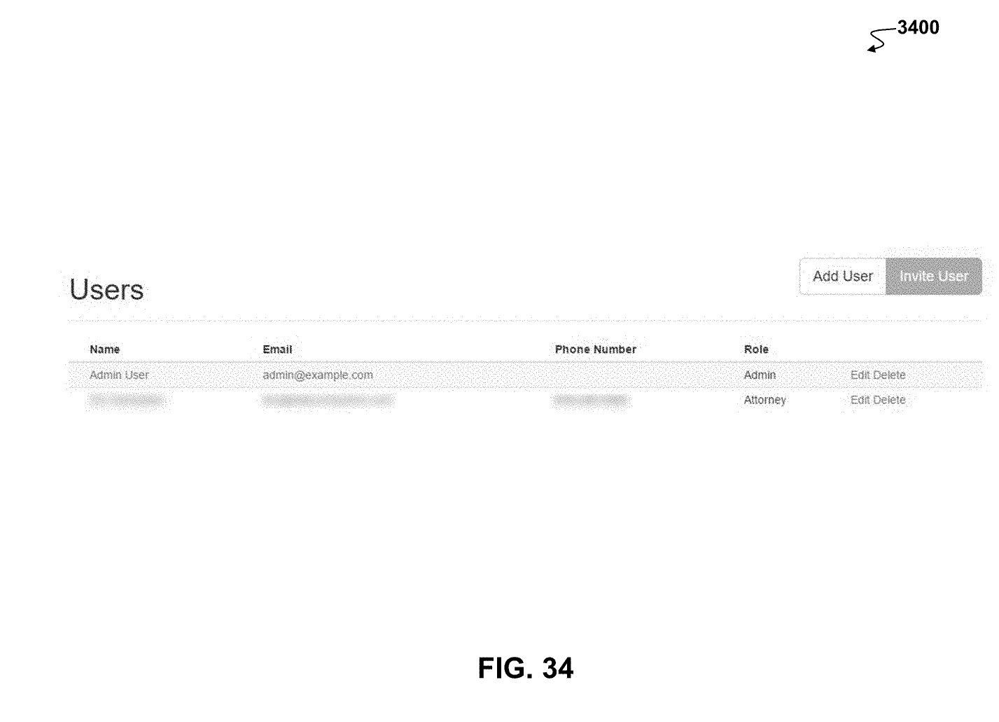

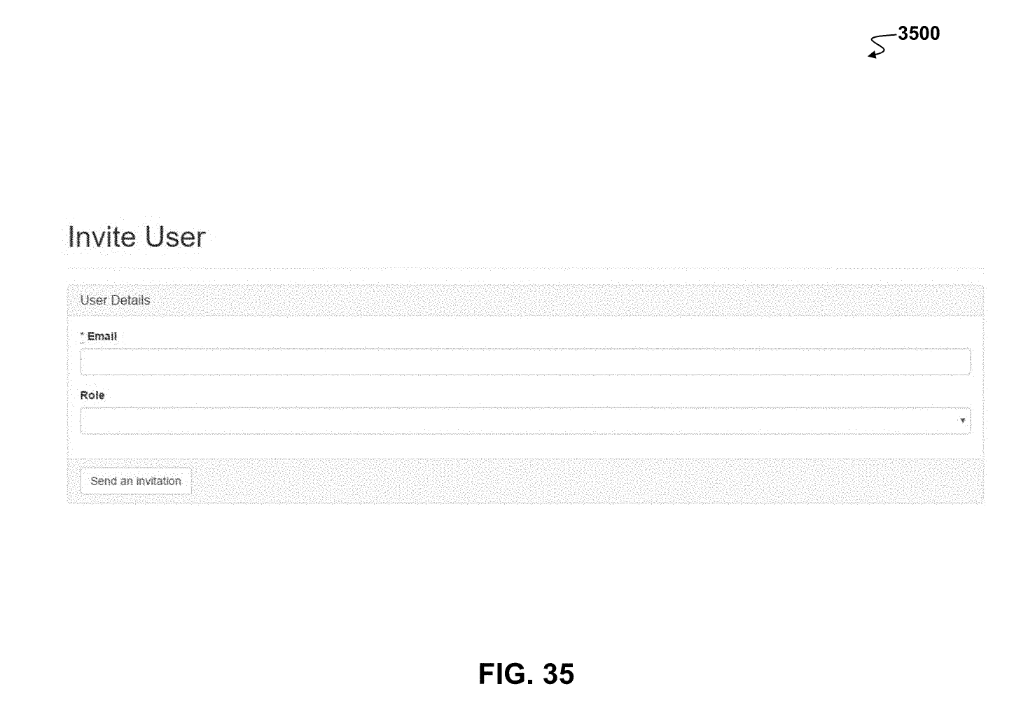

By way of example and not limitation, example screens of a direct access system for patient medical records user access subsystem utilizing a user account module 13, a user role module 14, and a user permission policy module 15 are depicted in FIGS. 34-35. In additional embodiments, new users may be added and assigned a user access role. In still additional embodiments, a role may include, but is not limited to, admins (who can access all aspects of the application), office managers (who can access most of the aspects of the application with the exception including, but not limited to, deleting reports), office users (who may access the application without being able to view edit or change any bill or report or deleting incidents, attorneys, practitioners, or patients), attorneys (who may be restricted to most of the aspects of the application with the exception of adding or viewing incidents), and physicians (who can also be restricted from most of the application with the exception of patient and incident modules).

In the user screen 3400 embodiment of FIG. 34, a user can add a new user by simply clicking on the white button labeled “Add User” on the top right corner of the “Users” page. In many embodiments, this can redirect the user to the “New User” form. The invite user screen 3500 embodiment in FIG. 35 allows a user to invite a new user by completing this form. This can send an email to the email address specified in the field asking the recipient to follow a link to create his/her user profile. The users screen may include a series of fields that can include, but is not limited to:
2a) Email—Input an email address for the person the user would like to invite to create a user profile.
2b) Role—Input the role the user would like them to sign up as.
2c) Send an Invitation—Simple click on the bottom white button labeled “Send an Invitation” to send out the email to the recipient.

The types of roles that the system can accommodate may include, but is not limited to:
1) Admin—Can access all aspects of the application.
2) Office Manager—Can access most of the aspects of the application. Restrictions include: Deleting a report.
3) Office User—Can access most of the aspects of the application. Restrictions include: Viewing, editing, changing status, or deleting any bill or report, deleting incidents, attorneys, practitioners, or patients.
4) Attorney—Restricted from most of the aspects of the application. Permissions Include: Creating and viewing patients and incidents.
5) Physician—Restricted from most of the aspects of the application. Permissions include: Creating, viewing and editing patients and incidents.
6) Doctor—Can access most of the aspects of the application. Restrictions include but are not limited to: Bills, Attorneys, Insurances, and Practitioners

### Log Subsystem

In many embodiments, a log subsystem 16 can provide the functionality to log all actions performed by users. In further embodiments, the log may only be accessed by a user with an “Admin” role. In many more embodiments, the log subsystem 16 can also have the functionality to email an administrator when certain functions are accessed in a particular module.

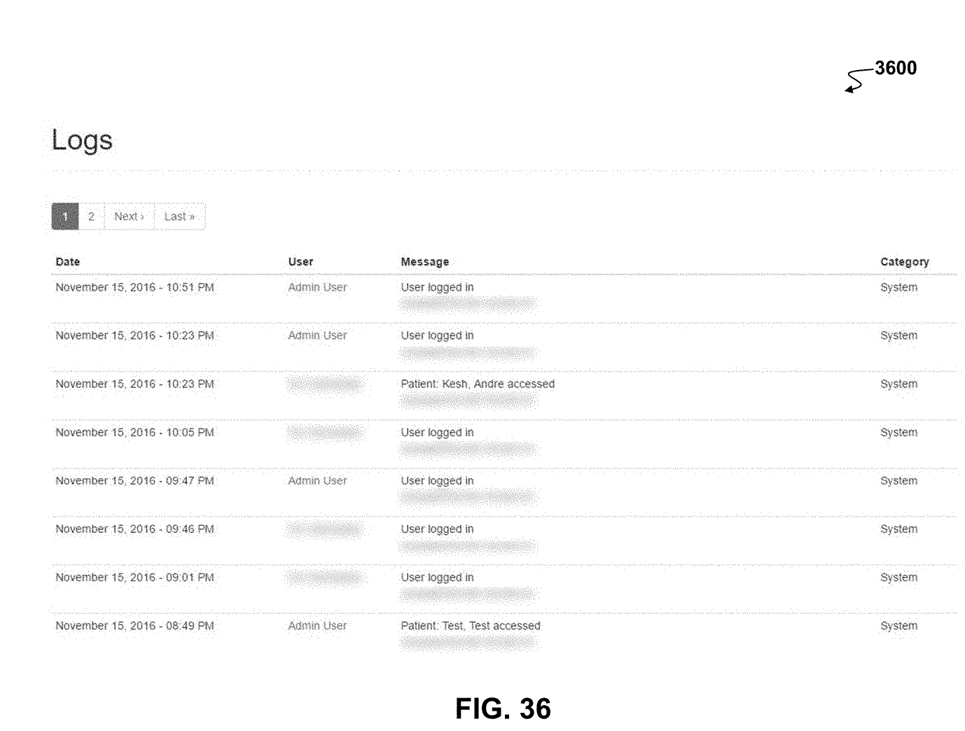

By way of example and not limitation, an example screen 3600 of a patient medical records system utilizing a log subsystem 16 is depicted in FIG. 36. In additional embodiments, the log subsystem 16 may record and store almost every piece of information available to the system in regards to changes made throughout the application. In certain embodiments, the log subsystem 16 may only be accessible by a user who has an admin user role.

### Front End Subsystem

In a number of embodiments, a frontend system 17 may provide a means of facilitating user input between the various modules of the direct access system and subsystems.

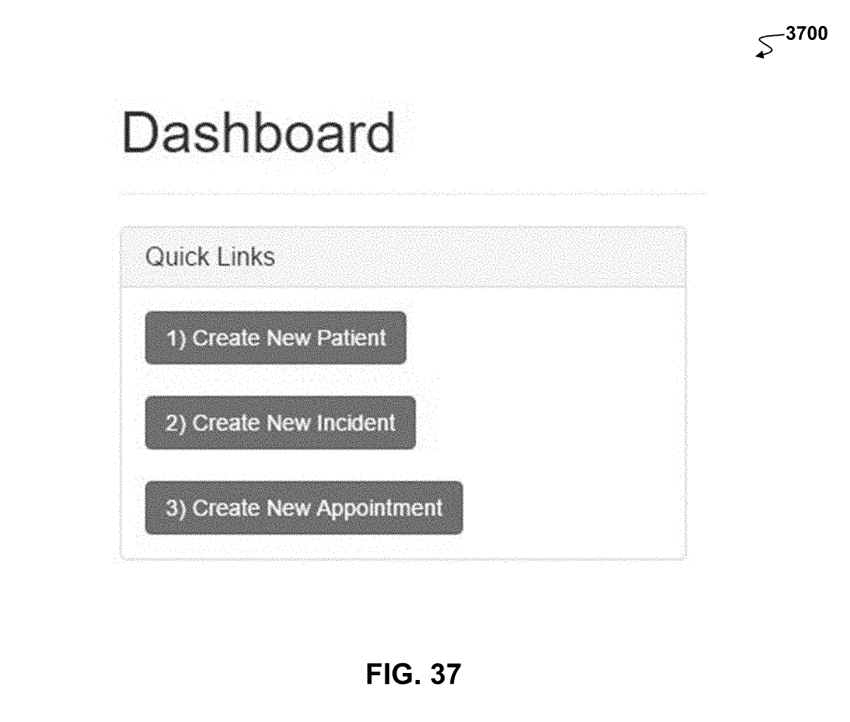

By way of example and not limitation, an example screen 3700 of a patient medical records system utilizing a front end subsystem 17 is depicted in FIG. 37. In certain embodiments, the front end subsystem may be adjusted and/or modified to present different options to a user based on their user role privileges such that only options that are allowed to the user are shown. When logging in as either an Attorney or a Physician, the user will be redirected to a role specific dashboard displayed in the embodiment of FIG. 37. The items displayed in the dashboard may include, but is not limited to:
1) Create New Patient—Create a new patient by simply clicking the first blue button labeled “Create New Patient”.
2) Create New Incident—Create a new incident by simply clicking the second blue button labeled “Create New Incident.”
3) Create New Appointment—Create a new appointment by simply clicking the third blue button labeled “Create New Appointment.”
4) Patients—Attorneys/Physicians can view a list of patients assigned to them via the “Patients” tab.
5) Status Update—View the latest action taken by the office for patients
6) Recent Reports—View the latest reports being processed by the office
7) Recent Bills—View the latest bills being processed by the office

In many embodiments, if the information entered for the patient already exists, it will not allow the user to make a duplicate. Attorneys may need to call the office of the specific location to make any changes to the patient profile. In a variety of embodiments, attorneys and/or physicians who create a patient or incident will be automatically assigned to that specific patient or incident. Also, in certain embodiments, attorneys who finalize the creation of a patient or incident can no longer edit the patient or incident. In additional embodiments, creating a new appointment can send a confirmation email to both the patient and the office of the location specified for the appointment.

### Accessing a Patient

With reference to FIG. 2, the present embodiments include a flowchart for accessing a patient. In many embodiments, the process 200 may begin by requesting 18 a patient's name. In certain embodiments, the process may begin via the patient information module 1. If the patient does not already exist 19, the process 200 may request 20 a patient's demographics and contact details. In certain embodiments, the system may request 21 the patient current doctors and then request 22 the patient's emergency contacts. In additional embodiments, the system may be able to scan 23 and upload any patient demographic documents that may be needed. In still additional embodiments, when all of the new patient information is entered and scanned, the system may then save 24 patient information in a database. In yet additional embodiments, when the process 200 finds that a requested 18 patients name does exist 19, the system may then check if the user is authorized 25 to access the selected record. In still yet additional embodiments, if the user is not authorized 25 to access the selected patient record, the process 200 will return 26 an access denied prompt and then return to request 18 a patient's name. In further additional embodiments, the user access subsystem may facilitate the authorization of access to the patient records. In more additional embodiments, the process 200 may retrieve 27 a record of a patient from a database when a user has been authorized 25 to access the record. In yet more additional embodiments, upon retrieval 27 of the record, the process 200 may generate 28 a display of the record for the user before ending the process 200.

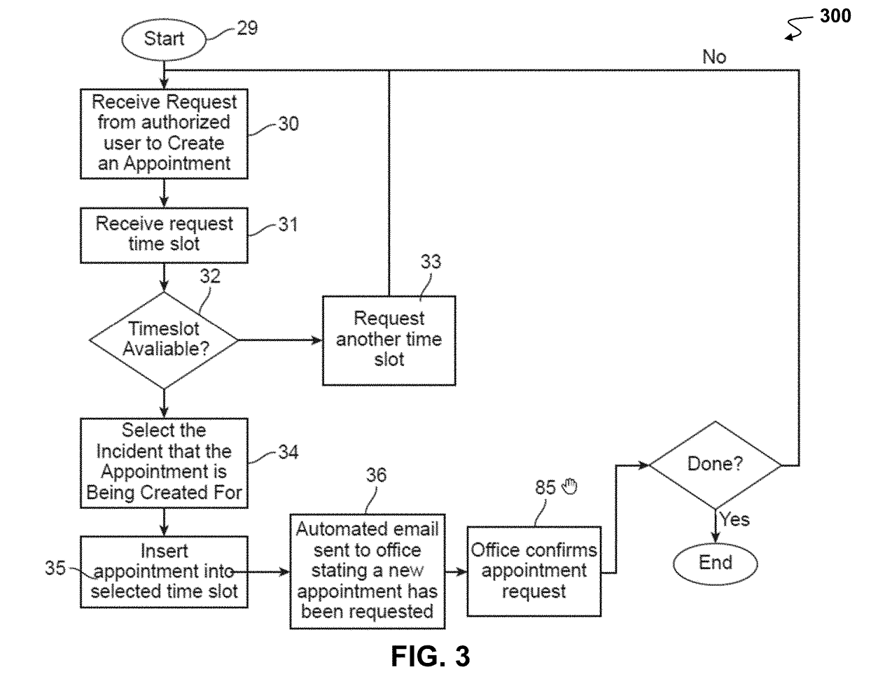

With reference to FIG. 3, the present embodiments include a flowchart for requesting an appointment. In a number of embodiments, the process 300 may begin 29 by receiving 30 a request from an authorized user to create an appointment. In a variety of embodiments, the user will generate a requested time slot that the system can then receive 31. In many embodiments, when the process 300 receives 31 a time slot request, the system will then verify if the time slot 32 is available. In certain embodiments where the time slot 32 is not available, the system can then request 33 another time slot before starting the process over. In the other embodiments when a time slot 32 is available, the process 300 can then provide a method for selecting 34 which incident the time slot is being created for. In more embodiments, the user may then insert 35 the appointment into a selected time slot. In still more embodiments, the process 300 can send out an automated 36 email to the office stating that a new appointment has been requested. In yet more embodiments, the office 85 can then confirm the appointment request as needed. The process 300 can then end if the user request has been completed or else start over again if the process 300 has not concluded.

With reference to FIG. 4, the present embodiments include a flowchart for creating a report. In many embodiments, the process 400 may begin by the user access subsystem when there is verification if the logged 37 in user has an assigned access specified as an admin or office manager. In the certain embodiments where the user does not have sufficient access, the system can return 38 an access denied message and start the process over. In the other embodiments where the user does have sufficient role access, the process 400 can then access 39 the patient record from a database and then retrieve 40 questions needed to generate a report for the patient from the database. In response to answering 41 the questions from the template, performing an exam, and formulate a diagnosis and plan of care, the process 400 can then save 42 the results to the database. In additional embodiments, the report management subsystem may facilitate this data saving and template generation. In still additional embodiments, the process can provide for the ability to review 43 and edit a generated report based off of the saved 42 results to the database. In yet additional embodiments, the user can then finalize 44 and approve the report before the system generates 45 a PDF of the report. In certain embodiments, in response to a generated 45 report, the process 400 can send an automated 46 email to an attorney to inform them that the report is ready before ending the process.

With reference to FIG. 5, the present embodiments include a flowchart for processing intake documents. In a plurality of embodiments, the process 500 begins with having a new patient 47 added to the database. In more embodiments, a user may then access 48 a patient in the system and select 49 a document intake mode for that patient. In still more embodiments, a user may then select 50 which document the patient will be filing out and then in response, the system can generate 51 a document based on the values from the database to display to the patient. In many embodiments, the patient fills 52 out the document. In yet more embodiments, when a document has been filed 52 out, the system can then generate 53 a PDF of the document with the added information. In certain embodiments, the system can then save 54 the generated PDF and attach it to the patent record before marking 55 the document as complete, which ends the process 500.

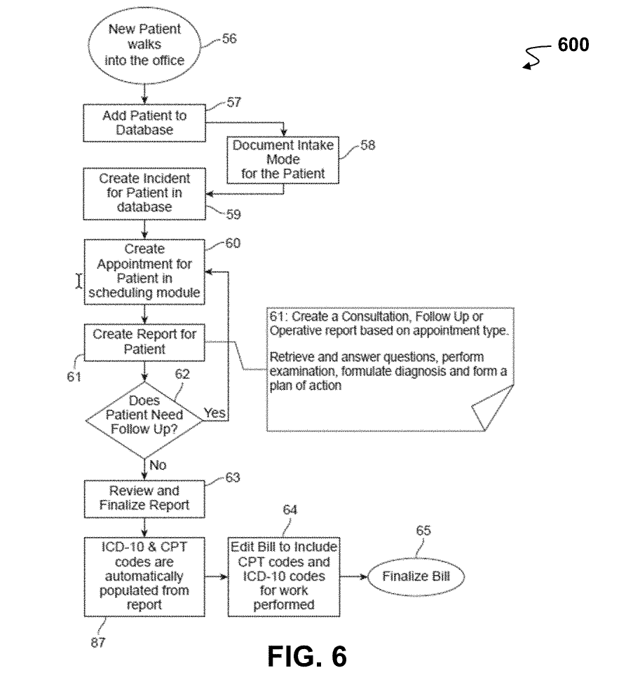

With reference to FIG. 6, the present embodiments include a flowchart for a general process overview of the medical records direct access system. In a number of embodiments, a new 56 patient may walk into the office. In additional embodiments, the system can then be used to add 57 a patient to the database which will then begin the document 58 intake mode for the patient. In still additional embodiments, when the patient has been entered into the system, an incident for the patient can be created 59 in the database. The process 600 can then in many embodiments, create 60 an appointment for the patient in the scheduling module. In response to the appointment being created the process 600 may, in certain embodiments, create 61 a report for the patient, who could create a consultation, follow up, or operative report based on the appointment type. In yet additional embodiments, the process 600 may determine if a patient 62 needs a follow up visit. In instances where a follow up visit is needed, an appointment can be created 60 for the patient in the scheduling module. In other embodiments, when no follow up is needed, the process 600 can allow a user to review 63 and finalize a report. In a variety of embodiments, the system can then populate ICD-10 87 and CPT codes automatically from the report. In more embodiments, the user may then edit 64 the bill to include CPT codes and ICD-10 codes for work performed before finalizing 65 the bill.

With reference to FIG. 7A, the present embodiments include a flowchart of an overview of an incident module. In a variety of embodiments, the incident module starts by accessing 66 the patient record and determining if an incident 67 for the patient is available. In further embodiments, when no incident 67 is available, the incident module process 700A may request 68 information about the details of the incident. In still further embodiments, the user can scan 69 and upload any imaging or doctor's report pertaining to the incident before saving 70 the information in the database. In yet further embodiments, a bill 71 can be automatically generated for the newly saved 70 incident. In certain embodiments, when an incident 67 for a patient is already saved, the process 700A may retrieve 72 the record of incident from the database. In most embodiments, the incident module will determine if a user is authorized 73 to access the incident record before generating 75 a display of the incident. In certain further embodiments, when the user is not authorized 73, the incident module can return 74 an access denied message to the user before starting the process over.

With reference to FIG. 7B, the present embodiments include a flowchart of an overview of a billing module. In a plurality of embodiments, the billing module process 700B can begin by determining if the logged 76 in user have the proper user role of admin or biller. In certain embodiments, when the user does not have the proper role, the process 700B can return 77 an access denied message to the user before starting the process 700B over again. When the logged 76 in user has the proper role, the biller 78 can receive a request to review and/or finalize a bill. In more embodiments, the billing module can allow access 79 to the bill association with the incident so the user may review 80 and edit the bill. In still more embodiments, the billing module may allow for the submission 81 of a bill for finalization. In certain embodiments, the bill 84 info can be sent to the accounts receivable database module. In other embodiments, the finalized bill can be used to generate 82 a PDF of the bill before an automated (83) email from the system may be sent to an attorney informing them that the bill is ready to be viewed, ending the process.

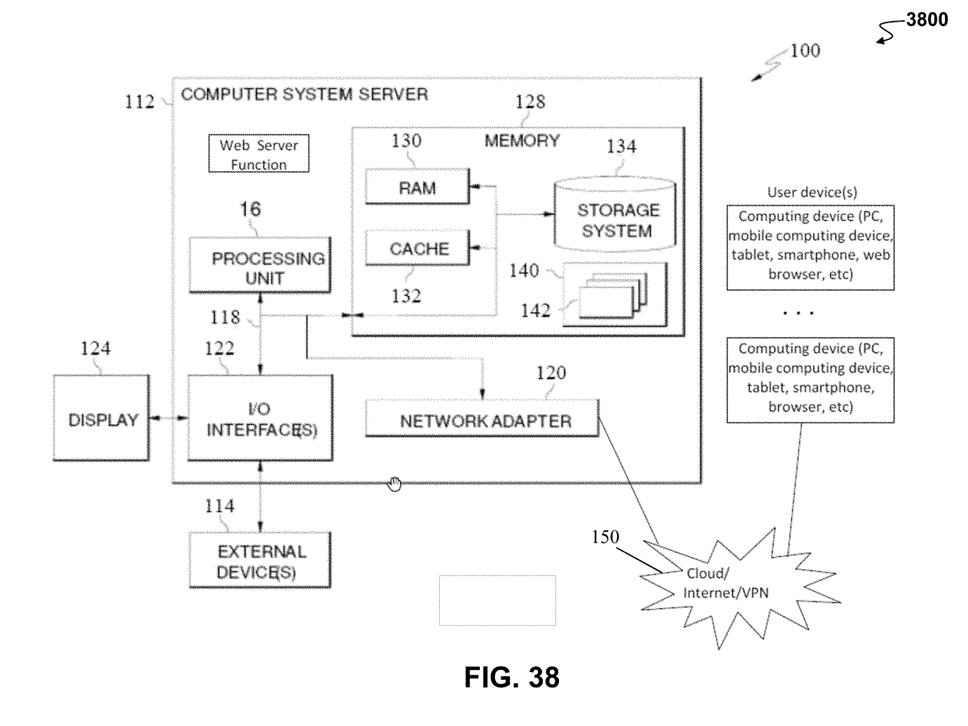

With reference to FIG. 38, the present embodiments include a medical records direct access systems server. The system 3800 shows a computer system/server 112 that may be in a cloud computing environment such as a computing node 100, shown in the form of a general-purpose computing device, though it can be a mobile computing device. The components of computer system/server 112 may include, but are not limited to, one or more processors or processing units 16, a system memory 128, and a bus 118 that couples various system components including system memory 128 to processor 16. Bus 118 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus. Computer system/server 112 typically includes a variety of computer system readable media. Such media may be any available media that is accessible by computer system/server 112, and it includes both volatile and non-volatile media, removable and non-removable media.

In one embodiment, user devices utilized for access to the server 112 (implementing the disclosed direct access system) may include PCs, mobile computing devices, tablets, smart phones with mobile GPS technology or other geolocation mechanism, etc. The server 112 and user devices can communicate with other devices, network, computing cloud, etc., via wired and/or wireless connections using communications modules.

System memory 128 can include computer system readable media in the form of volatile memory, such as random access memory (RAM) 130 and/or cache memory 132. Computer system/server 112 may further include other removable/non-removable, volatile/non-volatile computer system storage media. By way of example only, storage system 134 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 118 by one or more data media interfaces. The memory 128 may include at least one program product having a set (e.g., at least one) of program modules that are configured to carry out the functions of embodiments of the invention.

Program/utility 140, having a set (at least one) of program modules 142, may be stored in memory 128 by way of example, and not limitation, as well as an operating system, one or more application programs, other program modules, and program data. Each of the operating system, one or more application programs, other program modules, and program data or some combination thereof, may include an implementation of a networking environment. Program modules 142 generally carry out the functions and/or methodologies of embodiments of the invention as described herein. Computer system/server 112 may also communicate with one or more external devices 114 such as a keyboard, a pointing device, a display 124, etc.; one or more devices that enable a user to interact with computer system/server 112; and/or any devices (e.g., network card, modem, etc.) that enable computer system/server 112 to communicate with one or more other computing devices. Such communication can occur via Input/Output (I/O) interfaces 122. Still yet, computer system/server 112 can communicate with one or more networks such as a local area network (LAN), a general wide area network (WAN), and/or a public network (e.g., the Internet) via network adapter 120. As depicted, network adapter 120 communicates with the other components of computer system/server 112 via bus 118. It should be understood that although not shown, other hardware and/or software components could be used in conjunction with computer system/server 112. Examples, include, but are not limited to: microcode, device drivers, redundant processing units, external disk drive arrays, RAID systems, tape drives, and data archival storage systems, etc.

Cloud/Internet/VPN 150 may be any mixture of external network connections. Cloud computing is an emerging technology in the information technology (IT) industry. Cloud computing allows for the moving of applications, services and data from desktop computers back to a main server farm. The server farm may be off premises and be implemented as a service. By relocating the execution of applications, deployment of services, and storage of data, cloud computing offers a systematic way to manage costs of open systems, centralize information, and enhance robustness and reduce energy costs. The Internet is a global system of interconnected computer networks that can use the Internet protocol suite (TCP/IP) to link devices worldwide. The Internet is a network of networks that consists of private, public, academic, business, and government networks of local to global scope, linked by a broad array of electronic, wireless, and optical networking technologies. The Internet can carry a vast range of information resources and services, such as, but not limited to, inter-linked hypertext documents and applications of the World Wide Web (WWW), electronic mail, telephony, and file sharing. A virtual private network (VPN) extends a private network across a public network, and enables users to send and receive data across shared or public networks as if their computing devices were directly connected to the private network. Applications running across a VPN may benefit from increased functionality, security, and management of a private network.

VPNs may allow employees to securely access a corporate intranet while located outside the office. They are used to securely connect geographically separated offices of an organization, creating one cohesive network. Individual Internet users may secure their transactions with a VPN, to circumvent geo-restrictions and censorship, or to connect to proxy servers for the purpose of protecting personal identity and location in order to stay anonymous on the internet.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

The visual displays in the figures are generated by modules in local applications on computing devices and/or on the system/platform, and displayed on electronic displays of computing devices for user interaction and form graphical user interface for interaction with the system/platform disclosed herein.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The above description presents the best mode contemplated for carrying out the present embodiments, and of the manner and process of practicing them, in such full, clear, concise, and exact terms as to enable any person skilled in the art to which they pertain to practice these embodiments. The present embodiments are, however, susceptible to modifications and alternate constructions from those discussed above that are fully equivalent. Consequently, the present invention is not limited to the particular embodiments disclosed. On the contrary, the present invention covers all modifications and alternate constructions coming within the spirit and scope of the present disclosure. For example, the steps in the processes described herein need not be performed in the same order as they have been presented, and may be performed in any order(s). Further, steps that have been presented as being performed separately may in alternative embodiments be performed concurrently. Likewise, steps that have been presented as being performed concurrently may in alternative embodiments be performed separately.

What is claimed is:

1. A system for accessing patient medical records comprising:
a frontend system;
a log subsystem;
a direct access system, wherein the direct access system includes:
a content management subsystem for document intake creation and database storage;
a report management subsystem for setting appointments and generating reports;
a billing subsystem for generating billing and incidents reports; and
a user access subsystem for user account management and access restrictions.

2. The system of claim 1, wherein the content management subsystem further includes:
a patient information module;
a document intake module;
an attorney database module;
an insurance company database module; and
a physician database module.

3. The system of claim 1, wherein the report management subsystem further includes:
an incident information module;
a report template module;
an appointments module; and
a patent report module.

4. The system of claim 1, wherein the billing subsystem further includes:
a billing codes module;
an accounts receivables module;
an incident invoice module; and
a PDF generation module.

5. The system of claim 1, wherein the user access subsystem further includes:
a user account module;
a user role module; and
a user permission policy module.

6. The system of claim 2, wherein the document intake module:
adds a new patient to the database;
accesses the new patient;
selects a document intake mode for the new patient;
selects a document for the new patient to complete;
generates a document from a template based on the selected document;
displays the generated document;
provides a method of user input on the document on the display including a method of indicating completion;
generates a portable document format (PDF) document of the displayed document based upon a received response of completion;
store the generated PDF document in a patient medical record associated with the new patient;
mark the stored PDF document as complete in the in the patient medical record.

7. The system of claim 3, wherein the patient reporting module:
authorizes a user onto the system;
accesses a patient record from a database;
determines type of report to be generated;
generates questions for user based on determined type of report;
receives report answer data from user;
stores received report answer data to the database;
generates a preliminary report;
presents the generated preliminary report to the user;
generates a finalized report; and
transmits a message to a third party based upon the generated finalized report.
